(19) United States
(12) Patent Application Publication
(10) Pub. No.: US 2021/0040134 A1
(43) Pub. Date: Feb. 11, 2021

(54) COMBINATIONS OF MACROLIDE COMPOUNDS AND IMMUNE CHECKPOINT INHIBITORS

(71) Applicant: ISR IMMUNE SYSTEM REGULATION HOLDING AB (PUBL), Stockholm (JP)

(72) Inventor: Ola Winqvist, Uppsala (SE)

(21) Appl. No.: 17/040,680

(22) PCT Filed: Mar. 25, 2019
(86) PCT No.: PCT/EP2019/057364

§ 371 Date: Sep. 23, 2020

(30) Foreign Application Priority Data
Mar. 23, 2018 (EP) 18163703.4
Mar. 23, 2018 (EP) 18163705.9

(51) Int. Cl. C07H 17/08; C07K 16/28

(52) U.S. Cl. CPC C07H 17/08; A61K 45/06; C07K 16/2818

(57) ABSTRACT

The present invention provides a combination of immune stimulating macrolides with checkpoint inhibitors. The combinations have synergistic effects and can be used in treating viral diseases and cancer.

### FIELD OF THE INVENTION

The present invention relates to combinations of immune checkpoint inhibitors and macrolides capable of stimulating the immune system, named immunolides. The present invention relates to the combinations as such and to the combinations for use in medicine, notably in the immunotherapeutic treatment of cancer and in the treatment of viral diseases such as HIV.

### BACKGROUND OF THE INVENTION

Cancer cells are characterized by a myriad of genetic mutations and epigenetic alterations that give rise to a large variety of cancer-specific antigens. These antigens are detected by T cells, which utilize the antigens to distinguish precancerous and/or cancerous cells from their normal counterparts and elicit a cancer-specific immune response. The amplitude and quality of the T-cell-mediated immune response is normally regulated by immune checkpoints, which can be defined as stimulatory and inhibitory molecules and/or molecular pathways acting to increase or decrease, respectively, the magnitude of a response. Under normal physiological conditions, immune checkpoints are crucial for the prevention of autoimmunity and protection from tissue damage resulting from pathogenic infections. However, cancer cells may utilize dysregulation of immune checkpoint proteins as a way to obtain immune resistance.

One approach to trigger T cell-mediated antitumor immune responses has been termed “checkpoint blockade”, referring to the blockade or inhibition of immune-inhibitory checkpoints that are utilized by cancer cells. Since many immune checkpoints are initiated by ligand-receptor interactions, these checkpoints may be blocked by antibodies or modulated by recombinant forms of the ligands and/or receptors in question.

Several immune checkpoints, either alone or in combination, are relevant in terms of enhancing T cell-mediated antitumor immune responses. These include, but are not limited to, cytotoxic T-lymphocyte associated antigen 4 (CTLA4, also known as CD152), programmed cell death protein 1 (PD-1, also known as CD279), PD-1 ligand 1 (PD-L1, also known as B7-H1 and CD274), PD-1 ligand 2 (PD-L2, also known as B7-DC and CD-273), T-cell membrane protein 3 (TIM3, also known as HAVcr2), adenosine A2a receptor (A2aR), lymphocyte activation gene 3 (LAG3, also known as CD 223), and B7-H3 (also known as CD276), B7-H4 (also known as B7-S1, B7X, and VCTN1), 2B4 (also known as CD244), and B and T lymphocyte attenuator (BTLA, also known as CD272). Moreover, other examples of relevant immune checkpoints can be found in the scientific and patent literature and are also within the scope of the present invention.

Although immune checkpoint inhibition is useful for enhancing T cell-mediated anti-tumor immunity, it is contemplated by the present inventors that combining immune checkpoint inhibition with one or more complementary mechanisms to further enhance T cell activation will provide even better antitumor effects. To this end, the present inventors have realised that macrolides have immunostimulating anti-cancer and immunostimulating anti-viral effect, which have led the inventors to the present invention utilizing complementary mechanisms to achieve improved treatment regimens.

CD4⁺ T cells are key mediators of the immune response, and there is a great need in the art for methods and means of increasing the immune competence of CD4⁺ T cells in cancer patients.

### BRIEF DESCRIPTION OF THE DRAWINGS

FIG. 1. The structures of the macrolides Erythromycin A, Compound 1, Compound A, compound B and EM703.

FIG. 2. CD69 upregulation on T- and B-cells. PBMC were treated for 24 h with compound 1, compound A and activation controls LPS and IFN-gamma. The expression of the early activation marker CD69 was measured on the CD4⁺ T cell population (left) and CD19+ B cell population (right) with flow cytometry. Values represents mean fluorescent intensity, MFI, and error bars standard deviation in the triplicate samples.

FIG. 3. HLA-A,B,C upregulation on T- and B-cells. PBMC were treated for 24 h with compounds 1 or A and activation controls LPS and IFN-γ. The expression of HLAA,B,C was measured on the CD4+ T cell population (left) and CD19+ B cell population (right) with flow cytometry. Values represents mean fluorescent intensity, MFI, and error bars standard deviation in the triplicate samples.

FIG. 4. CD80 and HLA-DR upregulation on blood monocytes. PBMC were treated for 24 h with compounds 1 or A as well as activation controls LPS and IFN-gamma. The expression of CD80 and HLA-DR was measured on the monocyte cell population with flow cytometry. Values represents mean fluorescent intensity, MFI, and error bars standard deviation in the triplicate samples.

FIG. 5. CD80 upregulation on blood monocytes. PBMC were treated for 24 h with compounds 1 or A as well as activation control IFN-gamma. The expression of CD80 was measured on the monocyte cell population with flow cytometry. Values represents mean fluorescent intensity, MFI, and error bars standard deviation in the triplicate samples.

FIG. 6. Production of IL-10 from PBMCs after stimulation with compound 1 for 48 h or 1 week, measured with ELISA.

FIG. 7. CD4 T cell proliferation after 6 days stimulation with compound 1, measured with proliferation dye Celltrace violet (Invitrogen) and flow cytometry. Untreated cells (UNT) or compound A were used as controls.

FIG. 8. Upregulation of IL-7 receptor α (CD127) on CMV specific CD8 T cells after incubation with compound 1, measured with flow cytometry.

FIG. 9: Interferon-gamma secretion (as measured by cytometric bead assay) from PBMCs (from a CMV+ donor) grown with CMV peptides in the presence or absence of compound 1 or A for 5 days.

FIG. 10: Interferon-gamma secretion (as measured by cytometric bead assay) from macrophages stimulated with indicated compound for 48 h.

FIG. 11: Chemokine RANTES secretion (as measured by cytometric bead assay) from PBMC or macrophages stimulated with indicated compound for 48 h.

FIG. 12: IL12p70 secretion (as measured by cytometric bead assay) from PBMC or macrophages stimulated with indicated compound for 48 h.

FIG. 13: IL1 b secretion (as measured by cytometric bead assay) from PBMC, macrophages or CD4 T cells stimulated with indicated compound for 48 h.

FIG. 14: % CD25 high cells in blood of C57bl/6 mice injected 24 h previously with indicated dose of compound 1. CD25 expression was measured by flow cytometry.

FIG. 15: % MHC class I high CD11 b+ cells in spleen of 3 individual C57bl/6 mice injected 24 h previously with indicated compound. MHC class I and CD11 b expression was measured by flow cytometry.

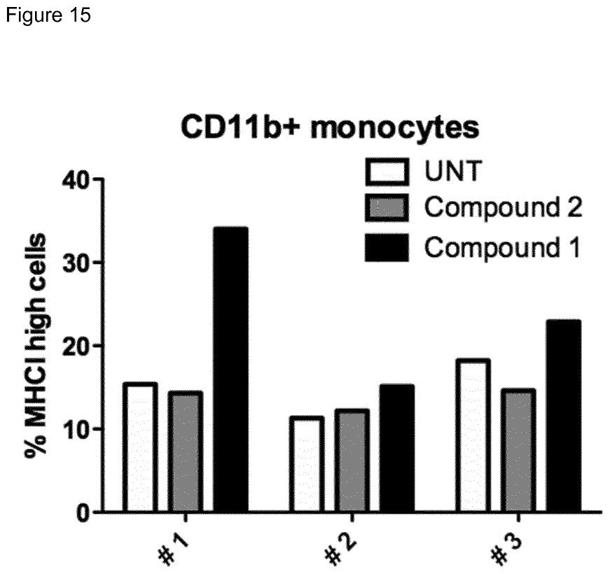

FIG. 16: Synergistic effect between anti-PD-1 blockade and ISR397. C57BL/6J mice were inoculated subcutaneously with B16-F10 melanoma cells and then treated with anti-PD-1 (closed circle), anti-PD-1+ISR397 (closed squares) or left untreated (closed triangles). Tumor volumes measured on day 3, 8, 11, 15, 18 are shown.

FIG. 17: Synergistic effect between anti-PD-1 blockade and ISR397. C57BL/6J mice were inoculated subcutaneously with B16-F10 melanoma cells and then either left untreated (pink), treated with anti-PD-1 (purple) or treated with anti-PD-1+ISR397 (red). Tumor volumes measured on termination of the experiment (day 18) are shown.

FIG. 18. Synergistic effect between anti-PD-1 blockade and ISR397. C57BL/6J mice were inoculated subcutaneously with B16-F10 melanoma cells and then treated with anti-PD-1 (closed circle), anti-PD-1+ISR397 (closed squares) or left untreated (closed triangles). Tumor volumes measured on day 3, 8, 11, 15, 18 are shown.

FIG. 19. Depiction of the proposed mechanism of ISR397 (compound 1) action.

### INTRODUCTION TO THE INVENTION

Macrolides, such as erythromycin and azithromycin, have been used for years in the treatment of bacterial infections. Erythromycin is a polyketide natural product macrolide produced by fermentation of the actinomycete Saccharopolyspora erythraea. Azithromycin is a semisynthetic azalide derivative of erythromycin. Many references exist describing the antibacterial activity of macrolides, such as erythromycin. This antibacterial mechanism is achieved through molecule binding to the P-site on the bacterial 50S bacterial ribosome, thus interfering with the tRNA binding.

Many references describe generation of analogues of erythromycin via semisynthesis and biosynthetic engineering. In particular, methods have been described for semisynthetic removal of the glycosyl groups on erythromycin, desosamine/cladinose and mycarose. Further methods have been described for biotransformation to add alternative glycosyl groups to the erythromycin aglycone (eg see Gaisser et al. 2000, Schell et al. 2008 and WO 2001/079520). The main focus of this published work, however, has been to generate antibacterial erythromycin analogues.

WO2007/004267 discloses methods and compositions for the treatment of a solid tumor by administering compositions comprising nanoparticles comprising an mTOR inhibitor and an albumin in combination with compositions comprising a second therapeutic agent.

WO2016/100882 discloses a combination comprising an immunomodulator and a second therapeutic agent for use in treating a cancer, wherein the immunomodulator is an inhibitor of an immune checkpoint molecule.

### DESCRIPTION OF THE INVENTION

The present invention relates to a combination of a macrolide and an immune checkpoint inhibitor to improved treatment especially in cancer and in cancers where stimulation of the immune system is beneficial.

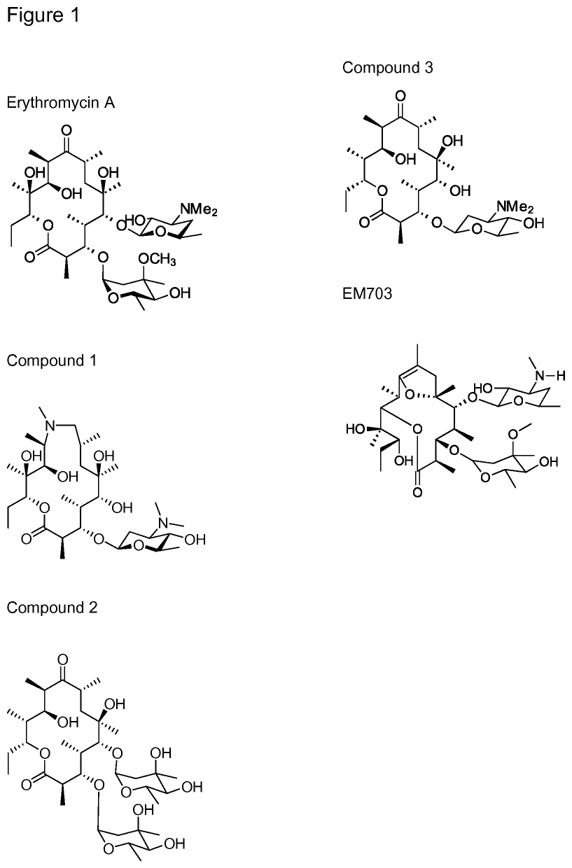

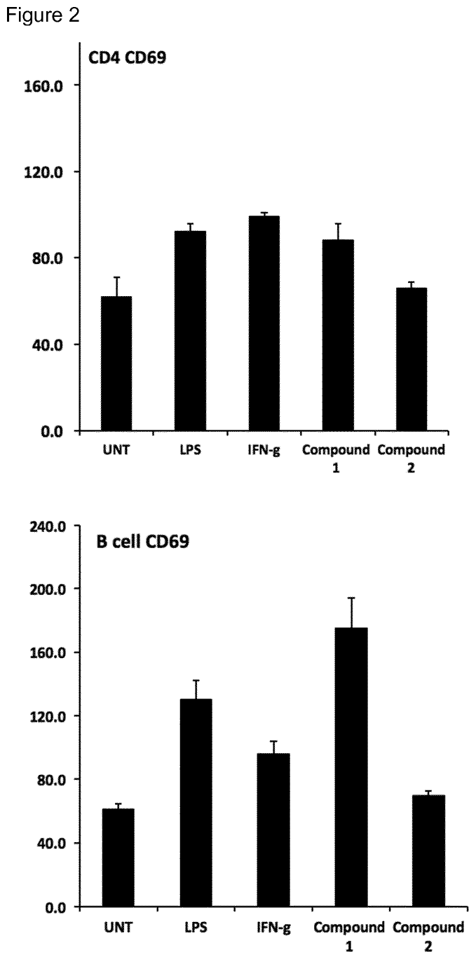

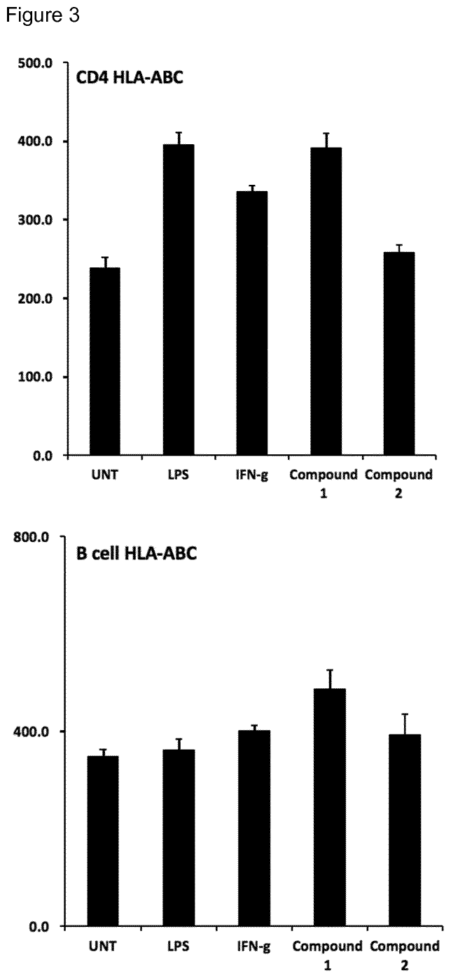

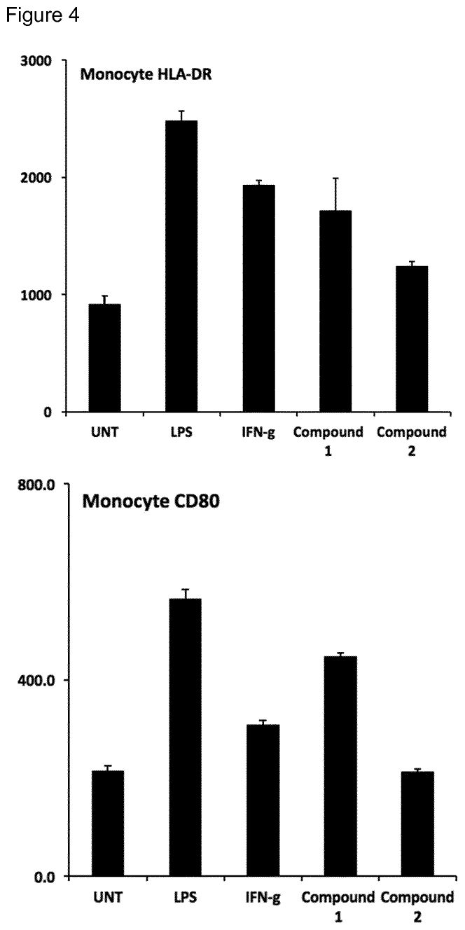

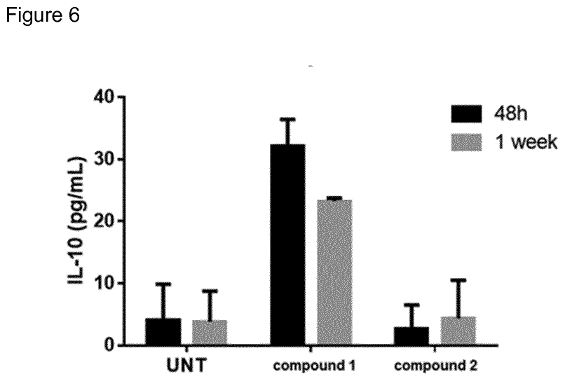

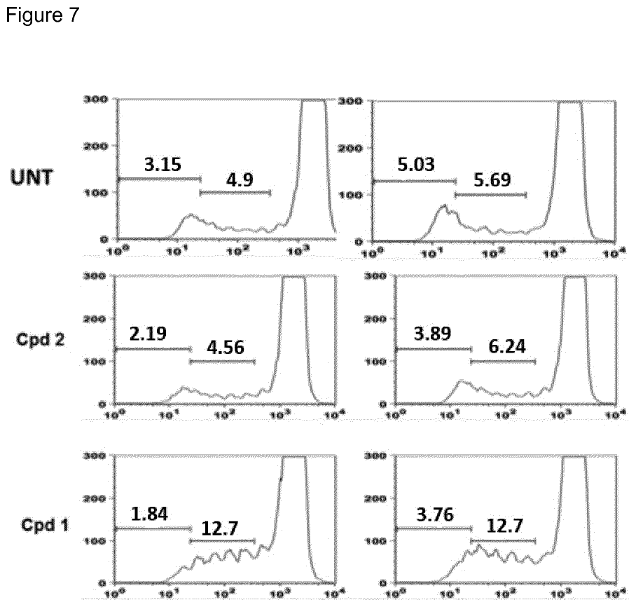

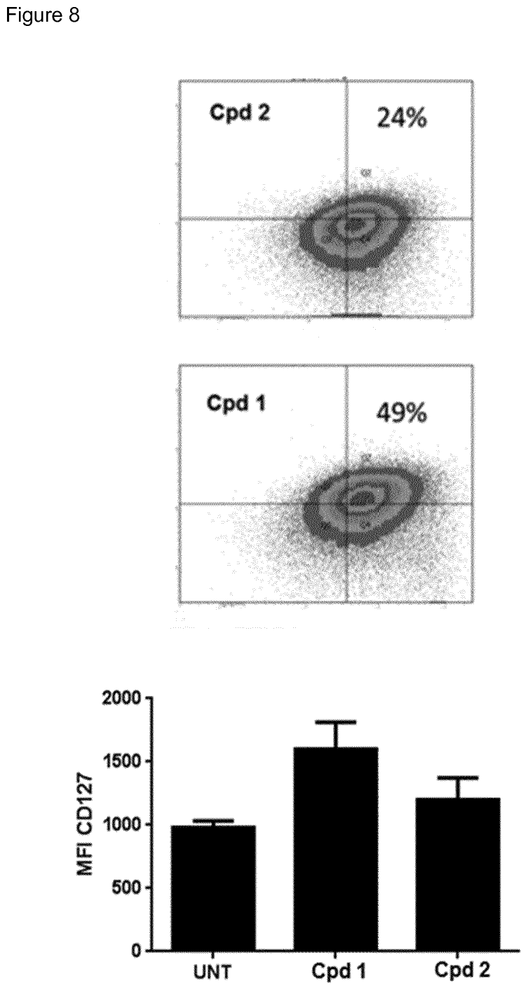

Immune stimulating activity from macrolides that lack antibacterial activity has previously not been reported. Surprisingly, it has now been found that compounds of the invention, such as compound 1 (FIG. 8) had a potent immune stimulating effect on several cell types of the immune system. After 24-48 h of in vitro stimulation of peripheral blood mononuclear cells (PBMC) with 1 μM compound 1, the activation marker CD69 was upregulated on CD4⁺ T cells and B cells (FIG. 1). We also observed upregulation of the MHC class I molecule (HLA-ABC) on T- and B-cells (FIG. 2), indicating an effect on antigen presentation of viral antigens. Stimulation of monocytes in the PBMC population with compound 1 led to the upregulation of the co-stimulatory molecule CD80 as well as the antigen presenting molecule MHC class II (HLA-DR) (FIG. 3). Monocytes differentiated into macrophages also exhibited CD80 upregulation in response to stimulation with compound 1 (FIG. 4). Furthermore, PBMCs stimulated with compound 1 expressed an altered cytokine profile with increased production of the immunosuppressive cytokine IL-10, indicating an immune inhibitory effect under certain conditions. Further analysis of the immunological effect of compound 1 revealed an altered cytokine driven proliferation profile of T cells after six days of stimulation, measured with flow cytometry (FIG. 6). In addition, virus-specific T cell proliferation was affected by compound 1. PBMCs from cytomegalovirus (CMV) infected donors cultured in the presence of CMV antigen and compound 1 displayed an altered phenotype of activated CMV-specific CD8⁺ T cells with an increased expression of IL-7 receptor α (CD127) (FIG. 7). CD127 is crucial for T cell homeostasis, differentiation and function, and reduced expression correlates with disease severity in HIV and other chronic viral diseases (Crawley et al. 2012).

In summary, compound 1 has a surprising ability to specifically activate and modify an immune response by affecting antigen presentation, co-stimulation and T cell activation and proliferation. In many of the examples presented herein, compound 2 (FIG. 8), another related macrolide erythromycin analogue with altered glycosylation previously published in Schell et al. 2008 (as compound 20), was included as negative control since it showed little or no activity in the assays.

The macrolides used in a combination with immune checkpoint inhibitors maximize the modulating effects of the immune system while minimizing the therapeutically unwanted direct antibacterial effects.

Thus, the present invention relates to a combination of a macrolide and an immune checkpoint inhibitor. The combination is useful for the prevention and treatment of cancer. It is contemplated that the combination of a macrolide and an immune checkpoint inhibitor will lead to an enhanced anti-tumor effect by combining the immune stimulating effect of the macrolide with the release of the break on the immune system mediated by the checkpoint inhibitor.

Macrolides useful for such combinations include macrolides of Formula (I) (see separate paragraph herein), but are not limited thereto. Specific immune checkpoint inhibitors of interest include agents selected from CTLA4 inhibitors, PD-1 inhibitors, PD-L1 inhibitors, PD-L2 inhibitors, LAG3 inhibitors, B7-H3 inhibitors, and CMTM6 inhibitors, but are not limited thereto. In a separate paragraph herein is given examples of immune checkpoint inhibitors suitable for use in combination with a macrolide.

Particularly interesting combinations of a macrolide and an immune checkpoint inhibitor include combinations, wherein the macrolide is selected from the compounds described herein. Of more particular interest are combinations of a macrolides selected from compounds given herein with structural formulas, including compound 1 (ISC397) and an immune checkpoint inhibitor selected from inhibitors of PD-1, PD-L1 and CTLA4 such as ISC397+PD-1, ISC397+PD-L1 or ISC397+CTLA-4 or ISC397+PD1+CTLA-4 or ISC397+PD-L1+CTLA-4.

The combination of a macrolide and an immune checkpoint inhibitor may be in the form a pharmaceutical composition comprising a macrolide, an immune checkpoint inhibitor, and one or more pharmaceutically acceptable excipients, or it may be in the form of two pharmaceutical compositions, with one composition comprising a macrolide and one or more pharmaceutically acceptable excipients and the other composition comprising an immune checkpoint inhibitor and one or more pharmaceutically acceptable excipients. In the latter case, the two compositions may be designed for the same or different administration route.

Alternatively, the combination of a macrolide and an immune checkpoint inhibitor may be in the form a cosmetic composition comprising a macrolide, an immune checkpoint inhibitor, and one or more cosmetically acceptable excipients.

The combination of a macrolide and an immune checkpoint inhibitor may further be in the form a pharmaceutical kit comprising in a single package:

i) a first composition comprising a macrolide,
ii) a second composition comprising an immune checkpoint inhibitor, and
iii) instructions for use.

### General Use of a Combination of the Invention

The combination of a macrolide and an immune checkpoint inhibitor is useful in medicine and/or cosmetics. The combination of a macrolide and an immune checkpoint inhibitor is of particular interest for use in medicine. Potential applications include methods of treatment or prevention of any relevant cancer form, the method comprising administering to a human or animal subject in need thereof a therapeutically effective amount of a combination of a macrolide and an immune checkpoint inhibitor.

The invention also relates to a method for treating or preventing cancer, the method comprising administering to a human or animal subject in need thereof a therapeutically effective amount of a combination according to any one of the claims and embodiments described herein.

The combination of a macrolide and an immune checkpoint inhibitor, including pharmaceutical compositions and pharmaceutical kits comprising said combination, are contemplated to be useful for the prevention and treatment of any form of cancer, including but not limited to Adrenal Cancer, Anal Cancer, Bile Duct Cancer, Bladder Cancer, Bone Cancer, Brain/CNS Tumors, Breast Cancer, Castleman Disease, Cervical Cancer, Colon/Rectum Cancer, Endometrial Cancer, Esophagus Cancer, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Acute Myeloid Leukemia, Chronic Lymphocytic Leukemia, Acute Lymphocytic Leukemia, Chronic Myeloid Leukemia, Chronic Myelomonocytic Leukemia, Liver Cancer, Non-Small Cell Lung Cancer, Small Cell Lung Cancer, Lung Carcinoid Tumor, Lymphoma, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Basal and Squamous Cell Skin Cancer, Melanoma, Merkel Cell Skin Cancer, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor.

The combinations of the invention disclosed herein may also be used to treat diseases, disorders, conditions, and symptoms, where immune response stimulation is useful, such as in treating patients infected with viral agents or with viral diseases such as HIV, Adenovirus, Alphavirus, Arbovirus, Borna Disease, Bunyavirus, Calicivirus, Condyloma Acuminata, Coronavirus, Coxsackievirus, Cytomegalovirus, Dengue fever virus, Contageous Ecthyma, Epstein-Barr virus, Erythema Infectiosum, Hantavirus, Viral Hemorrhagic Fever, Viral Hepatitis, Herpes Simplex Virus, Herpes Zoster virus, Infectious Mononucleosis, Influenza, Lassa Fever virus, Measles, Mumps, Molluscum Contagiosum, Paramyxovirus, Phlebotomus fever, Polyoma-virus, Rift Valley Fever, Rubella, Slow Disease Virus, Smallpox, Subacute Sclerosing Panencephalitis, Tumor Virus Infections, West Nile Virus, Yellow Fever Virus, Rabies Virus and Respiratory Syncitial Virus. Especially, HIV is of interest in the present context.

The macrolides as described herein can be used in medicine, medical research or in the manufacture of a composition for such use. Accordingly, when in the following the term “macrolides” is used in connection with medical use or pharmaceutical composition, the term is intended also to include the compounds of Formula (I). In particular, medical use as described herein of the macrolides of Formula (I) includes compounds, wherein when R₁ is Et, R₂ is a sugar of Formula (II), R₁₃ is OH, R₁₄ is H, Rₐ is H, R₄ is Me, R₅ is H, R₆ is OH, R₇ is H, R₈ is NR₁₁R₁₂, R₉ is H, R₁₀ is H, and X is C═O.

The macrolides of Formula (I) are designed in order to minimize direct antibacterial effects, but rather focus on immune activating properties. When a compound of the invention is added to cultures of bacteria E. coli, S. salivarius, L. casei, B. longum or M. luteus, no or minimal antibacterial effect is recognized. The advantage of having compounds with isolated immune stimulatory properties that affect the host cells is that development of bacterial resistance is avoided. In addition, the well-known side effect of macrolides affecting the gut microbiota, with the risk of overgrowth of Clostridium difficile causing diarrhea and pseudomebraneous colitis, is avoided. Many viruses and cancers have developed mechanisms to avoid immune recognition, i.e. by down regulating HLA expression to avoid detection by T cells. The mechanism of the compounds of the intervention relies on the activation and increased expression of HLA molecules on infected cells. HLA molecules load and present peptides derived from intracellular infectious agents in order to present a recognition signal for T cells allowing elimination of infected cells.

The advantageous properties of the compounds of Formula (I) compared with known macrolides may include one or more of the following:
Reduced direct antibacterial activity
Improved MHC class I stimulation
Improved immunomodulation
Improved activation of antigen presenting cells
Improved T-cell response
Improved anti tumoral response
Improved antiviral activity
Improved MHC class II antigen presentation

### Pharmaceutical Compositions Comprising the Combinations of the Invention

The present invention also provides pharmaceutical compositions comprising the combinations of the invention together with one or more pharmaceutically acceptable diluents or carriers.

The combination of a macrolide and an immune checkpoint inhibitor may be in the form a pharmaceutical composition comprising a macrolide, an immune checkpoint inhibitor, and one or more pharmaceutically acceptable excipients, or it may be in the form of two pharmaceutical compositions, with one composition comprising a Macrolide and one or more pharmaceutically acceptable excipients and the other composition comprising an immune checkpoint inhibitor and one or more pharmaceutically acceptable excipients. In the latter case, the two compositions may be designed for the same or different administration route.

The combinations of the invention or formulation thereof may be administered by any conventional route, for example but without limitation they may be administered parenterally, orally, topically or via a mucosa (including buccal, sublingual, transdermal, vaginal, rectal, nasal, ocular, etc.), via a medical device (e.g. a stent), or by inhalation. The treatment may consist of a single administration or a plurality of administrations over a period of time.

Each compound (i.e. macrolide and checkpoint inhibitor, respectively) or composition comprising a compound may be administered by separate administration routes and in different formulation types. Moreover, the administration frequency may not be the same.

The dosage regimen of the macrolides and the checkpoint inhibitors may be varied depending on the properties of the compound or composition in question. The dosage regimen may consist of a single administration of the combination or of two compositions each comprising either the macrolide or the checkpoint inhibitor. The dosage regime may also be a plurality of administrations over one or more periods of time. Administration may be once daily, twice daily, three times daily, four times daily, less frequently, or more frequently, depending on the specific use, the disease to be treated, and the physical condition and characteristics (such as gender, weight, and age) of the patient to be treated. The treatment may also be by continuous administration such as e.g. intravenous administration via a drop or via depots or sustained-release formulations.

Whilst it is possible for the combination of the invention to be administered as such, it is preferable to present it as a pharmaceutical formulation, together with one or more acceptable carriers. The carrier(s) must be “acceptable” in the sense of being compatible with the compound of the invention and not deleterious to the recipients thereof. Examples of suitable carriers are described in more detail below.

The pharmaceutical compositions may conveniently be presented in a suitable dosage form including a unit dosage form and may be prepared by any of the methods well known in the art of pharmacy. Such methods include the step of bringing into association the compound of the invention with one or more excipients. In general, the pharmaceutical compositions are prepared by uniformly and intimately bringing into association the compound of the invention with the excipient(s), and then, if necessary, shaping the resulting composition into e.g. a tablet.

The combinations of the invention will normally be administered by any conventional administration route normally by the oral or any parenteral route, in the form of pharmaceutical formulations comprising the active ingredients, optionally in the form of a nontoxic organic, or inorganic, acid, or base, addition salt, in a pharmaceutically acceptable dosage form. Depending upon the disorder and patient to be treated, as well as the route of administration, the compositions may be administered at varying doses and/or frequencies.

The pharmaceutical compositions must be stable under the conditions of manufacture and storage; thus, if necessary should be preserved against the contaminating action of microorganisms such as bacteria and fungi. In case of liquid formulations such as solutions, dispersion, emulsions and suspensions, the carrier can be a solvent or dispersion medium containing, for example, water, ethanol, polyol (e.g. glycerol, propylene glycol and liquid polyethylene glycol), vegetable oils, and suitable mixtures thereof.

For example, the combinations of the invention may be administered orally, buccally or sublingually in the form of tablets, capsules, films, ovules, elixirs, solutions, emulsions or suspensions, which may contain flavouring or colouring agents.

Pharmaceutical compositions of the present invention suitable for oral administration may be presented as discrete units such as capsules, cachets or tablets, each containing a predetermined amount of the active ingredients; as multiple units e.g. in the form of a tablet or capsule: as a powder or granules; as a solution or a suspension in an aqueous liquid or a non-aqueous liquid; or as an oil-in-water liquid emulsion or a waterin-oil liquid emulsion. The active ingredient may also be presented as a bolus, electuary or paste.

Solutions or suspensions of the combinations of the invention suitable for oral administration may also contain one or more solvents including water, alcohol, polyol etc. as well as one or more excipients such as pH-adjusting agent, stabilizing agents, surfactants, solubilizers, dispersing agents, preservatives, flavors, etc. Specific examples include e.g. N,N-dimethylacetamide, dispersants e.g. polysorbate 80, surfactants, and solubilisers, e.g. polyethylene glycol, Phosal 50 PG (which consists of phosphatidylcholine, soya-fatty acids, ethanol, mono/diglycerides, propylene glycol and ascorbyl palmitate). The formulations according to the present invention may also be in the form of emulsions, wherein a combination of the invention may be present in an emulsion such as an oil-in-water emulsion or a water-in-oil emulsion. The oil may be a natural or synthetic oil or any oil-like substance such as e.g. soy bean oil or safflower oil or combinations thereof.

Tablets may contain excipients such as microcrystalline cellulose, lactose (e.g. lactose monohydrate or lactose anhydrous), sodium citrate, calcium carbonate, dibasic calcium phosphate and glycine, butylated hydroxytoluene (E321), crospovidone, hypromellose, disintegrants such as starch (preferably corn, potato or tapioca starch), sodium starch glycollate, croscarmellose sodium, and certain complex silicates, and granulation binders such as polyvinylpyrrolidone, hydroxypropylmethylcellulose (HPMC), hydroxypropylcellulose (HPC), macrogol 8000, sucrose, gelatin and acacia. Additionally, lubricating agents such as magnesium stearate, stearic acid, glyceryl behenate and talc may be included.

A tablet may be made by compression or moulding, optionally with one or more accessory ingredients. Compressed tablets may be prepared by compressing in a suitable machine the active ingredients in a free-flowing form such as a powder or granules, optionally mixed with a binder (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredients therein using, for example, hydroxypropylmethylcellulose in varying proportions to provide desired release profile.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the combinations of the invention may be combined with various sweetening or flavouring agents, colouring matter or dyes, with emulsifying and/or suspending agents and with diluents such as water, ethanol, propylene glycol and glycerin, and combinations thereof.

Pharmaceutical compositions of the invention suitable for topical administration in the mouth include lozenges comprising the active ingredients in a flavoured basis, usually sucrose and acacia or tragacanth; pastilles comprising the active ingredients in an inert basis such as gelatin and glycerin, or sucrose and acacia; and mouth-washes comprising the active ingredients in a suitable liquid carrier.

Pharmaceutical compositions of the invention adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollient in creams or ointments and ethanol or oleyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the compound, in sufficient quantities to produce a cream or ointment having the desired consistency.

Pharmaceutical compositions of the invention adapted for transdermal administration may be presented as discrete patches intended to remain in intimate contact with the epidermis of the recipient for a prolonged period of time. For example, the active ingredients may be delivered from the patch by iontophoresis.

For applications to external tissues, for example the mouth and skin, the compositions are preferably applied as a topical ointment or cream. When formulated in an ointment, the active ingredients may be employed with either a paraffinic or a water-miscible ointment base.

Alternatively, the active ingredients may be formulated in a cream with an oil-in-water cream base or a water-in-oil base.

For parenteral administration, fluid unit dosage forms are prepared utilizing the active ingredients and a sterile vehicle, for example but without limitation water, alcohols, polyols, glycerine and vegetable oils, water being preferred. The active ingredients, depending on the vehicle and concentration used, can be either colloidal, suspended or dissolved in the vehicle. In preparing solutions the active ingredients can be dissolved in water for injection and filter sterilised before filling into a suitable vial or ampoule and sealing.

Advantageously, agents such as local anaesthetics, preservatives and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability.

Parenteral suspensions are prepared in substantially the same manner as solutions, except that the active ingredients are suspended in the vehicle instead of being dissolved and sterilization cannot be accomplished by filtration. The active ingredients can be sterilised by exposure to ethylene oxide before suspending in the sterile vehicle. Advantageously, a surfactant or wetting agent is included in the composition to facilitate uniform distribution of the active ingredients.

It should be understood that in addition to the ingredients particularly mentioned above the formulations of this invention may include other agents conventional in the art having regard to the type of formulation in question, for example those suitable for oral administration may include flavouring agents. A person skilled in the art will know how to choose a suitable formulation and how to prepare it (see eg Remington's Pharmaceutical Sciences 18 Ed. or later). A person skilled in the art will also know how to choose a suitable administration route and dosage.

It will be recognized by one of skill in the art that the optimal quantity and spacing of individual dosages of a combination of the invention will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

All % values mentioned herein are % w/w unless the context requires otherwise.

### Macrolides for Use in the Combination of the Invention

The immune stimulating macrolides for use in a combination of the invention are macrolides of Formula (I) or pharmaceutically acceptable salts hydrates, solvates, tautomers, enantiomers or diastereomers thereof:

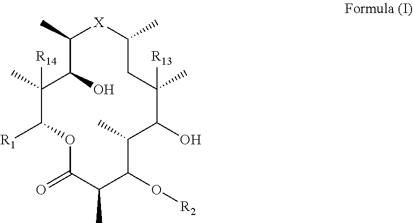

wherein X is selected from C═O, —NR₃CH₂—, —CH₂NR₃—, —NR₃(C═O)—, —(C═O)NR₃—, C═NOH, and —CH(OH)—, and R₂ is a sugar of Formula (II) or Formula (III):

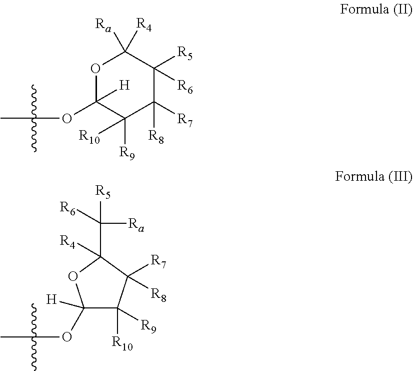

wherein R₁ is selected from an alkyl, heteroalkyl, cycloalkyl, aryl, and heteroaryl moiety, wherein alkyl moiety is selected from C₁-C₆ alkyl groups that are optionally branched, wherein heteroalkyl moiety is selected from C₁-C₆ alkyl groups that are optionally branched or substituted and that optionally comprise one or more heteroatoms, wherein cycloalkyl moiety is selected from a C₁-C₆ cyclic alkyl groups that are optionally substituted and that optionally comprise one or more heteroatoms, wherein aryl moiety is selected from optionally substituted C₆ aromatic rings, wherein heteroaryl moiety is selected from optionally substituted C₁-C₅ aromatic rings comprising one or more heteroatoms,
wherein heteroatoms are selected from O, N, P, and S,
wherein substituents, independently, are selected from alkyl, OH, F, Cl, NH₂, NH-alkyl, NH-acyl, S-alkyl, S-acyl, O-alkyl, and O-acyl,
wherein acyl is selected from C₁-C₄ optionally branched acyl groups,
wherein R₃ is selected from H and Me,
wherein R₄ is selected from H and Me,
wherein Rₐ is selected from H and —CR₂₁R₂₂R₂₃,
wherein R₂₁, R₂₂, R₂₃, and R₅, R₆, R₇, R₈, R₉, and R₁₀, independently, are selected from H, Me, NR₁₁R₁₂, NO₂, and OR₁₁,
wherein R₂₃ together with R₄ in Formula (II), R₄ together with R₅ in Formula (II), R₅ together with R₇ in Formula (II), and R₇ together with R₉ in Formula (II), independently, may be joined to represent a bond to leave a double bond between the carbon atoms that each group is connected to, so that
wherein if R₂₃ and R₄ are joined to forma double bond, then Formula (II) can be represented by:

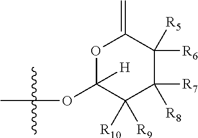

wherein if R₄ and R₅ are joined to form a double bond, then Formula (II) can be represented by:

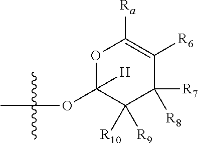

wherein if R₅ and R₇ are joined to form a double bond, then Formula (II) can be represented by:

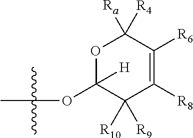

wherein if R₇ and R₉ are joined to form a double bond, then Formula (II) can be represented by:

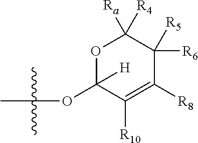

wherein R₄ together with R₅ in Formula (III), R₄ together with R₇ in Formula (III), and R₇ together with R₉ in Formula (III), independently, may be joined to represent a bond to leave a double bond between the carbon atoms that each group is connected to, so that
wherein if R₄ and R₅ are joined to form a double bond, then Formula (III) can be represented by:

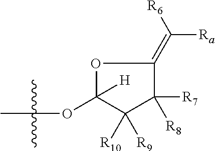

wherein if R₄ and R₇ are joined to form a double bond, then Formula (III) can be represented by:

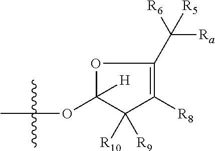

wherein if R₇ and R₉ are joined to form a double bond, then Formula (III) can be represented by:

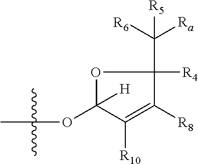

wherein R₂₁ together with R₂₂, R₅ together with R₆, R₇ together with R₈, or R₉ together with R₁₀ may be replaced with a carbonyl,
wherein R₁₁ and R₁₂, independently, are selected from H and alkyl,
wherein R₁₃ is selected from H, OH, and OCH₃,
wherein R₁₄ is selected from H and OH,
and wherein one of R₅, R₆, R₇, R₈, R₉ or R₁₀ is selected from NR₁₁R₁₂ and NO₂.

In some aspects, the macrolide is according to formula (i) with the proviso that when R₁ is Et, R₂ is a sugar of Formula (II), R₁₃ is H or OH, R₁₄ is H or OH, Rₐ is H, R₄ is Me, R₅ is H, R₆ is OH, R₇ is H, R₈ is NR₁₁R₁₂, R₉ is H, and R₁₀ is H, X may not be C═O,

with the proviso that when R₁ is Et, R₂ is a sugar of Formula (II), R₁₃ is H or OH, R₁₄ is H or OH, Rₐ is H, R₄ is Me, R₅ is OH, R₆ is H, R₇ is OH, R₈ is Me, R₉ is H, and R₁₀ is H, X may not be C═O,
with the proviso that when R₁ is Et, R₂ is a sugar of Formula (II), R₁₃ is H or OH, R₁₄ is H or OH, Rₐ is H, R₄ is Me, R₅ is OH, R₆ is H, R₇ is H, R₈ is NR₁₁R₁₂, R₉ is H, and R₁₀ is OH, X may not be C═O.

The immune stimulating macrolides of Formula (I) or pharmaceutically acceptable salts hydrates, solvates, tautomers, enantiomers or diastereomers thereof may have

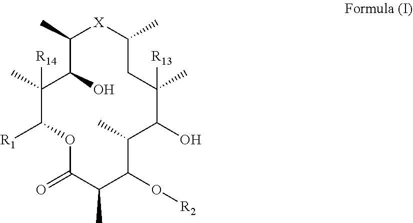

wherein X is selected from C═O, —NR₃CH₂—, and —CH(OH)—, and R₂ is a sugar of Formula (II):

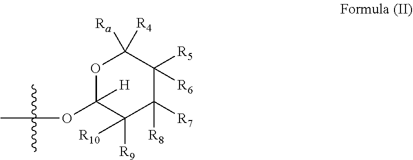

wherein R₁ is selected from and alkyl or cycloalkyl moiety,
wherein alkyl moiety is selected from C₁-C₆ alkyl groups that are optionally branched and, independently, optionally hydroxylated,
wherein cycloalkyl moiety is selected from C₁-C₆ optionally substituted cyclic alkyl groups,
wherein substituents are selected from alkyl and OH,
wherein R₃ is selected from H and Me,
wherein R₄ is selected from H and Me,
wherein Rₐ is selected from H and —CR₂₁R₂₂R₂₃,
wherein R₂₁, R₂₂, R₂₃, and R₅, R₆, R₇, R₈, R₉, and R₁₀, independently, are selected from H, Me, NR₁₁R₁₂, NO₂, and OR₁₁,
wherein R₂₃ together with R₄ in Formula (II), R₄ together with R₅ in Formula (II), R₅ together with R₇ in Formula (II), and R₇ together with R₉ in Formula (II), independently, may be joined to represent a bond to leave a double bond between the carbon atoms that each group is connected to, so that
wherein if R₂₃ and R₄ are joined to forma double bond, then Formula (II) can be represented by:

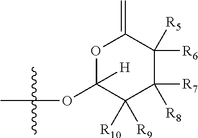

wherein if R₄ and R₅ are joined to form a double bond, then Formula (II) can be represented by:

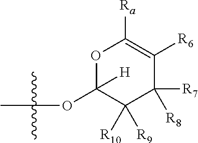

wherein if R₅ and R₇ are joined to form a double bond, then Formula (II) can be represented by:

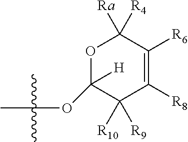

wherein if R₇ and R₉ are joined to form a double bond, then Formula (II) can be represented by:

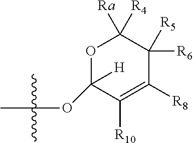

wherein R₂₁ together with R₂₂, R₅ together with R₆, R₇ together with R₈, or R₉ together with R₁₀ may be replaced with a carbonyl,
wherein R₁₁ and R₁₂, independently, are selected from H and alkyl,
wherein R₁₃ is selected from H, OH, and OCH₃,
wherein R₁₄ is selected from H and OH,
and wherein one of R₅, R₆, R₇, R₈, R₉ or R₁₀ is selected from NR₁₁R₁₂ and NO₂.

In an aspect, the above-mentioned macrolides are according to formula (I) with the proviso that when R₁ is Et, R₂ is a sugar of Formula (II), R₁₃ is H or OH, R₁₄ is H or OH, Rₐ is H, R₄ is Me, R₅ is H, R₆ is OH, R₇ is H, R₈ is NR₁₁R₁₂, R₉ is H, and R₁₀ is H, X may not be C═O.

with the proviso that when R₁ is Et, R₂ is a sugar of Formula (II), R₁₃ is H or OH, R₁₄ is H or OH, Rₐ is H, R₄ is Me, R₅ is OH, R₆ is H, R₇ is OH, R₈ is Me, R₉ is H, and R₁₀ is H, X may not be C═O.
with the proviso that when R₁ is Et, R₂ is a sugar of Formula (II), R₁₃ is H or OH, R₁₄ is H or OH, Rₐ is H, R₄ is Me, R₅ is OH, R₆ is H, R₇ is H, R₈ is NR₁₁R₁₂, R₉ is H, and R₁₀ is OH, X may not be C═O.

The macrolides may be provided by a method for producing a compound of formula (I), which involves addition of an aglycone with formula IV to a culture of a biotransformation strain which glycosylates at the 3-hydroxyl position.

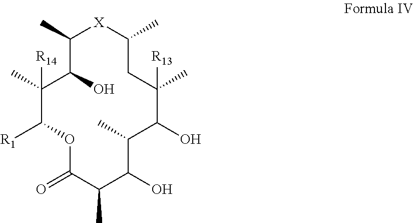

An interesting selection of macrolides are compounds wherein R₂ is selected from L-daunosamine, L-acosamine, L-ristosamine, D-ristosamine, 4-oxo-L-vancosamine, L-vancosamine, D-forosamine, L-actinosamine, 3-epi-L-vancosamine, L-vicenisamine, Lmycosamine, D-mycosamine, D-3-N-methyl-4-O-methyl-L-ristosamine, D-desosamine, N,N-dimethyl-L-pyrrolosamine, L-megosamine, L-nogalamine, L-rhodosamine, D-angolosamine, L-kedarosamine, 2′-N-methyl-D-fucosamine, 3-N,N-dimethyl-L-eremosamine, D-ravidosamine, 3-N,N-dimethyl-D-mycosamine/D-mycaminose, 3-N-acetyl-D-ravidosamine, 4-O-acetyl-D-ravidosamine, 3-N-acetyl-4-O-acetyl-D-ravidosamine, D-glucosamine, N-acetyl-D-glucosamine, L-desosamine, D-amosamine, D-viosamine, Lavidinosamine, D-gulosamine, D-allosamine, and L-sibirosamine.

Yet another interesting selection of macrolides are compounds wherein R₂ is selected from D-angolosamine, N-desmethyl D-angolosamine, N-didesmethyl D-angolosamine, N-desmethyl N-ethyl D-angolosamine, and N-didesmethyl N-diethyl D-angolosamine.

Yet another interesting selection of macrolides are compounds wherein R₂ is selected from N-desmethyl D-angolosamine, N-didesmethyl D-angolosamine, N-desmethyl N-ethyl D-angolosamine, and N-didesmethyl N-diethyl D-angolosamine.

Yet another interesting selection of macrolides are compounds wherein R₂ is a sugar according to Formula (II).

Yet another interesting selection of macrolides are compounds wherein R₂ is a sugar according to formula 2 wherein Rₐ is H, R₄ is Me, R₅ is H, R₆ is OH, R₇ is H, R₈ is NR₁₁R₁₂, R₉ is H and R₁₀ is H.

Yet another interesting selection of macrolides are compounds wherein R₁₁ is selected from H, Me, and Et, and R₁₂ is selected from H, Me, and Et.

Yet another interesting selection of macrolides are compounds wherein R₁₁ is Et and R₁₂ is Et.

Yet another interesting selection of macrolides are compounds wherein R₁₁ is Me and R₁₂ is Et.

Yet another interesting selection of macrolides are compounds wherein X is selected from C═O, —NR₃CH₂— and —CH(OH)—

Yet another interesting selection of macrolides are compounds wherein R₁ is selected from Me, Et, and cycloalkyl.

Yet another interesting selection of macrolides are compounds wherein R₁ is selected from Me and Et.

Yet another interesting selection of macrolides are compounds wherein X is selected from —NR₃CH₂— or —CH₂NR₃—.

Yet another interesting selection of macrolides are compounds wherein one of R₅, R₆, R₇, or R₈, is NR₁₁R₁₂.

Yet another interesting selection of macrolides are compounds wherein R₂₁, R₂₂, R₂₃, and R₅, R₆, R₇, R₈, R₉, and R₁₀, independently, are selected from H, Me, NR₁₁R₁₂, and OR₁₁.

Yet another interesting selection of macrolides are compounds wherein R₁₃ and R₁₄ are OH.

Of particular interest are macrolides of Formula (I), wherein R₁ is Et, R₂ is a sugar of Formula (II), R₁₃ is OH, R₁₄ is H, Rₐ is H, R₄ is Me, R₅ is H, R₆ is OH, R₇ is H, R₈ is NR₁₁R₁₂, R₉ is H, R₁₀ is H, and X is C═O.

Specific macrolides include:

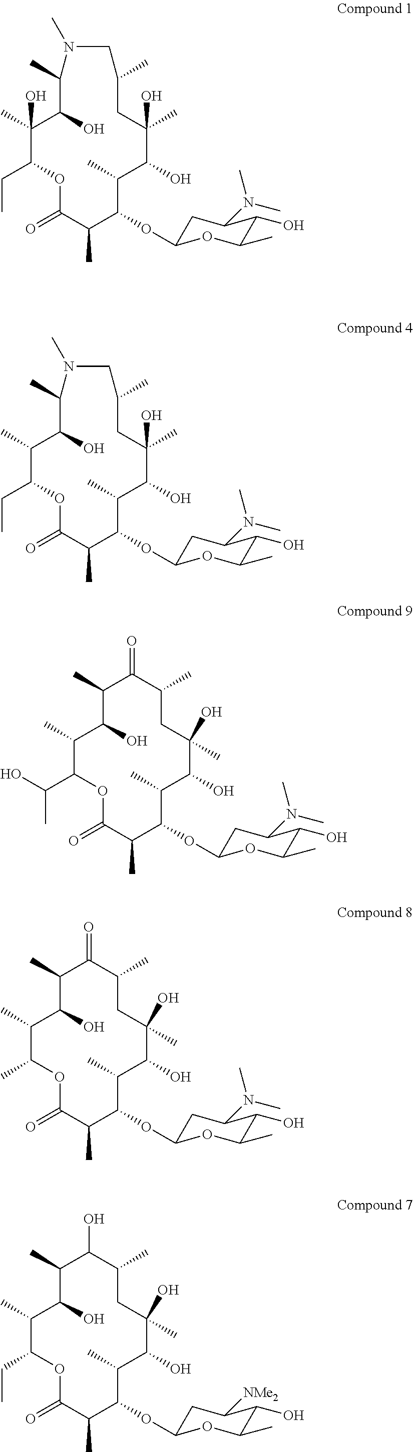

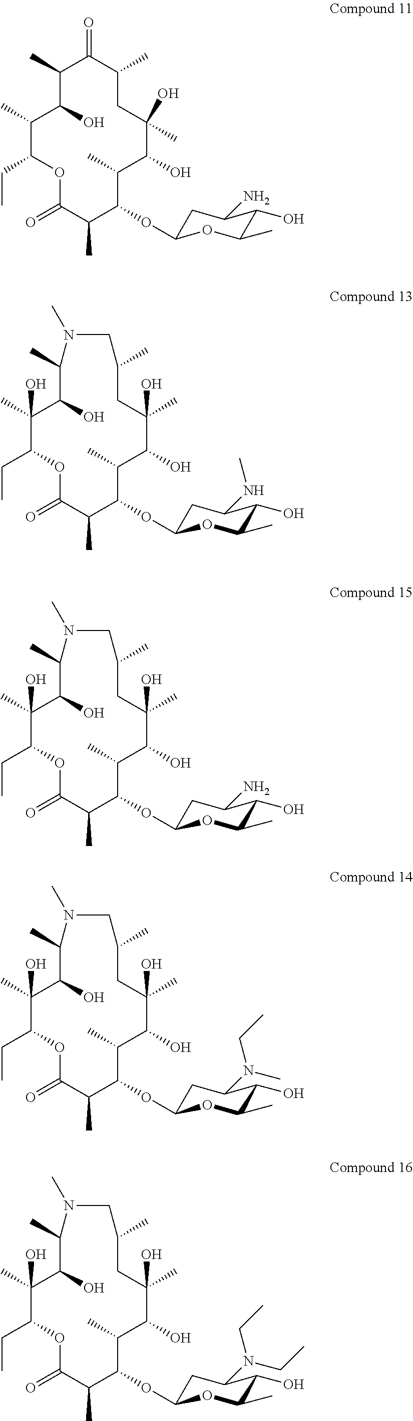

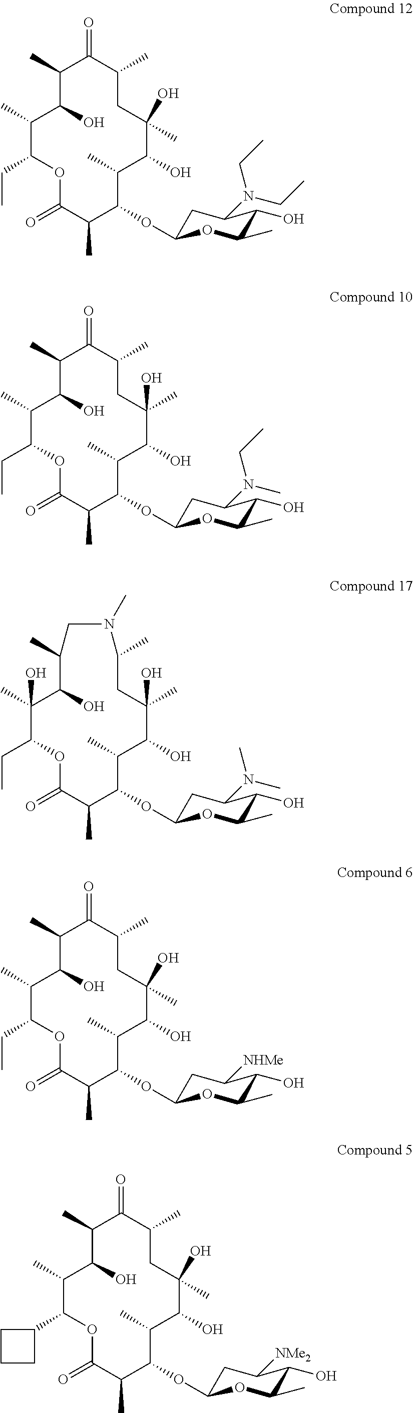

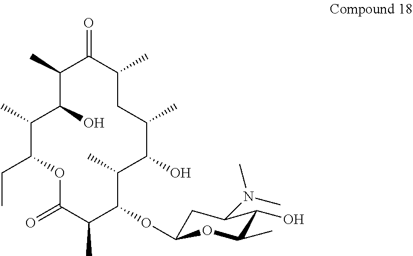

As seen from the examples herein some of the macrolides are without substantial antibacterial activity as defined herein.

### General Preparation Methods for Macrolides of Formula (I)

The skilled person will recognise that macrolides of Formula (I) may be prepared, using known methods, in a variety of ways. The routes below are merely illustrative of some methods that can be employed for the preparation of compounds of Formula (I).

Where an aglycone is required for biotransformation these can be accessed in a number of ways. Azithromycin and erythromycin are readily available and considered suitable starting points. The mycarose/cladinose and/or desosamine are removed by chemical methods, such as glycoside cleavage. Briefly, in one method the sugars may be removed by treatment with acid. In order to facilitate removal of the amino sugar it is first necessary to oxidise the dimethylamine to form an N-oxide which is then removed by pyrolysis. The resulting 5-O/3-O sugars can then be removed by acidic degradation. A suitable method is taught by LeMahieu et al. 1974 and Djokic et al. 1988. Finally, the compound is biotransformed using a bacterial strain which adds the amino sugar.

Another route to suitable aglycones is by fermentation and isolation from a suitable blocked mutant. For example, erythronolide B (3a) can be generated by fermentation of strains of S. erythraea blocked in glycosylation, such as strains and processes described, for example, in U.S. Pat. No. 3,127,315 (e.g. NRRL2361, NRRL2360, NRRL2359 and NRRL2338), Gaisser et al. 2000 (e.g. S. erythraea DM ΔBV ΔCIII). Briefly, the fermentation is conducted by methods known in the art. Typically, a seed culture is prepared and transferred to a production vessel. The production phase is between 4 and 10 days and the organism is grown between 24° C. and 30° C. with suitable agitation and aeration. The aglycone can then be isolated by extraction and purification.

Where an aglycone or compound of the invention possesses an amino sugar or any other tertiary amine and is prepared by fermentation, it will be necessary to extract the bacterial broth and purify the compound. Typically, the bacterial broth is adjusted to between pH 8 and 10, ideally 9.5. The broth can then be extracted with a suitable organic solvent. This solvent not be water miscible and is ideally ethyl acetate, methyl tert-butyl ether (MTBE) or solvents with similar properties. The broth and the solvent are mixed, ideally by stirring, for a period of time, e.g. 30 minutes or 1 hour. The phases are then separated and the organic extracts removed. The broth can be extracted in this manner multiple times, ideally two or three times. The combined organic extracts can then be reduced in vacuo. The residue is then dissolved or suspended in mildly acidic aqueous solvent. Typically, this is an ammonium chloride aqueous solution. This is then extracted with a water-immiscible organic solvent, such as ethyl acetate, a number of times, ideally 2 or 3 times. The resulting aqueous layer is collected and the pH is adjusted to between pH 8 and 10, ideally 9.0. The resultant aqueous layer is then extracted with a water-immiscible organic solvent, such as ethyl acetate, a number of times, ideally 2 or 3 times. The organic extracts are combined and reduced in vacuo to yield a crude extract enhanced in the target compound requiring further purification.

Compound purification can be done by chromatography or (re)crystallisation, and the methods required are well known to a person skilled in the art. Where chromatography is required on normal phase silica and an aglycone or compound of the invention possesses an amino sugar or other tertiary amine, then it is beneficial to add a basic modifier to the mobile phase. For instance, chromatography on normal phase silica can use a hexane, ethyl acetate, methanol system for elution with 0-5% aqueous ammonium hydroxide added. Ideally, 2% aqueous ammonium hydroxide is added. Following biotransformation, both unused aglycone and compound of the invention can be purified separately from the same crude extract using a suitable solvent system. If further purification is required, this may optionally be carried out by preparative HPLC.

Reductive amination to alkylate a primary or secondary amine is well known to a person skilled in the art. The amine is mixed in a solvent with an aldehyde or ketone and a reducing agent is added. Sodium borohydride can then reduce the imine or hemiaminal that results from the reaction of the amine and carbonyl, resulting in e.g. an alkylated amine. Sodium borohydride may also reduce other carbonyl groups present, e.g. ketones. In cases where a ketone also exists, it is preferred to use a reducing agent that is more specific to a protonated imine, such as sodium cyanoborohydride, though it will be obvious to a person skilled in the art that different reducing agents, solvents, temperatures, and reaction times may need to be tested to find the optimal conditions.

### Check-Point Inhibitors for Use in Combinations of the Invention

The presently known check-point inhibitors are of interest in connection with the present invention as well as still unidentified check-point inhibitors. Thus, of interest are agents selected from CTLA4 inhibitors such as ipilimumab and tremelimumab, or selected from PD-1 inhibitors such as pembrolizumab (MK3475), nivolumab (MDX-1106), pidilizumab (CT-011), AMP-224, or selected from PD-L1 inhibitors such as atezolizumab, avelumab, durvalumab, MDX-1105, Anti-PD-1 (clone RMP 1-14 from Merck, Johnson, Roche or Astra), or selected from PD-L2 inhibitors, or selected from LAG3 inhibitors such as IMP321, or selected from B7-H3 inhibitors such as enoblituzumab and MGD009, or selected from CMTM6.

Of particular interest are immune checkpoint inhibitors selected from ipilimumab, pembrolizumab, nivolumab, atezolizumab, avelumab, and durvalumab.

Of even more particular interest are immune checkpoint inhibitors selected from inhibitors of PD-1.

However, other examples of immune checkpoint inhibitors can be found in the scientific and patent literature and are also within the scope of the present invention.

### Definitions

The articles “a”, “an”, and “the” are used herein to refer to one or to more than one (i.e. at least one) of the grammatical objects of the article. By way of example “an analogue” means one analogue or more than one analogue.

As used herein the term “direct antibacterial effect” refers to the antibacterial activity of erythromycin and analogues which occurs through binding to the bacterial rRNA complex. This effect does not require presence of any host immune system components and therefore is apparent in standard antibacterial assays such as in vitro Minimum Inhibitory Concentration (MIC) assays and disk inhibition assays.

As used herein the term “without substantial antibacterial activity” is intended to mean that the compound of the invention has a MIC value of >64 μg/ml when tested in accordance with Example 13 herein for its antibacterial activity in E. coli, S. salivarius, L. casei and B. longum.

As used herein the term “immunostimulator” is intended to mean a compound that activates the immune system.

As used herein the sentence “immune checkpoint inhibitor targets an immune checkpoint” is intended to mean that it blocks checkpoint signalling.

As used herein the term “alkyl” refers to any straight or branched chain composed of only sp3-hybridized carbon atoms, fully saturated with hydrogen atoms such as e.g. —CₙH₂ₙ₊₁ for straight chain alkyls, wherein n can be in the range of 1 and 6 such as e.g. methyl, ethyl, propyl, isopropyl, n-butyl, isobutyl, sec-butyl, tert-butyl, n-pentyl, neopentyl, isopentyl, hexyl or isohexyl. The alkyl as used herein may be further substituted.

The term “heteroalkyl” in the present context designates a group —X—C-₁₋₆ alkyl used alone or in combination, wherein C₁₋₆ alkyl is as defined above and X is O, S, NH or N-alkyl. Examples of linear heteroalkyl groups are methoxy, ethoxy, propoxy, butoxy, pentoxy and hexoxy. Examples of branched heteroalkyl are iso-propoxy, sec-butoxy, tert-butoxy, iso-pentoxy and iso-hexoxy. Examples of cyclic heteroalkyl are cyclopropyloxy, cyclobutyloxy, cyclopentyloxy and cyclohexyloxy. The heteroalkyl as used herein may be further substituted.

As used herein the term “cycloalkyl” refers to a cyclic/ring structured carbon chains having the general formula of —CₙH₂ₙ₋₁ where n is between 3-6, such as e.g. cyclopropyl, cyclobytyl, cyclopentyl or cyclohexyl and the like. The cycloalkyl as used herein may be further substituted or contain a heteroatom (O, S, NH or N-alkyl) in the cyclic structure.

The term “aryl” as used herein is intended to include carbocyclic aromatic ring systems. Aryl is also intended to include the partially hydrogenated derivatives of the carbocyclic systems enumerated below.

The term “heteroaryl” as used herein includes heterocyclic unsaturated ring systems containing one or more heteroatoms selected among nitrogen, oxygen and sulphur, such as furyl, thienyl, pyrrolyl, and is also intended to include the partially hydrogenated derivatives of the heterocyclic systems enumerated below.

The terms “aryl” and “heteroaryl” as used herein refers to an aryl, which can be optionally unsubstituted or mono-, di- or tri substituted, or a heteroaryl, which can be optionally unsubstituted or mono-, di- or tri substituted. Examples of “aryl” and “heteroaryl” include, but are not limited to, phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), phenanthrenyl, fluorenyl, pentalenyl, azulenyl, biphenylenyl, thiophenyl (1-thienyl, 2-thienyl), furyl (1-furyl, 2-furyl), furanyl, thiophenyl, isoxazolyl, isothiazolyl, 1,2,3-triazolyl, 1,2,4-triazolyl, pyranyl, pyridazinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl (thianaphthenyl), indolyl, oxadiazolyl, isoxazolyl, quinazolinyl, fluorenyl, xanthenyl, isoindanyl, benzhydryl, acridinyl, benzisoxazolyl, purinyl, quinazolinyl, quinolizinyl, quinolinyl, isoquinolinyl, quinoxalinyl, naphthyridinyl, phteridinyl, azepinyl, diazepinyl, pyrrolyl (2-pyrrolyl), pyrazolyl (3-pyrazolyl), 5-thiophene-2-yl-2H-pyrazol-3-yl, imidazolyl(1-imidazolyl, 2-imidazolyl, 4-imidazolyl, 5-imidazolyl), triazolyl (1,2,3-triazol-1-yl, 1,2,3-triazol-2-yl, 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), isoquinolyl (1-isoquinolyl, 3-isoquinolyl, 4-isoquinolyl, 5-isoquinolyl, 6-isoquinolyl, 7-isoquinolyl, 8-isoquinolyl), quinolyl (2-quinolyl, 3-quinolyl, 4-quinolyl, 5-quinolyl, 6-quinolyl, 7-quinolyl, 8-quinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl)), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl)), indolyl (1-indolyl, 2-indolyl, 3-indolyl, 4-indolyl, 5-indolyl, 6-indolyl, 7-indolyl), indazolyl (1-indazolyl, 2-indazolyl, 3-indazolyl, 4-indazolyl, 5-indazolyl, 6-indazolyl, 7-indazolyl), benzimidazolyl, (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl). Non-limiting examples of partially hydrogenated derivatives are 1,2,3,4-tetrahydronaphthyl, 1,4-dihydronaphthyl, pyrrolinyl, pyrazolinyl, indolinyl, oxazolidinyl, oxazolinyl, oxazepinyl and the like.

The pharmaceutically acceptable salts of the compound of the invention include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts.

The invention is further described by the following non-limiting embodiments:

1. A combination of a macrolide and an immune checkpoint inhibitor.
2. The combination according to embodiment 1 wherein the macrolide is without substantial antibacterial activity.
3. The combination according to embodiment 2 wherein the macrolide has a MIC value of >64 μg/ml when tested according to the antibacterial activity test described in Example 13.
4. The combination of a macrolide and an immune checkpoint inhibitor according to any of the preceding embodiments wherein the macrolide has Formula I

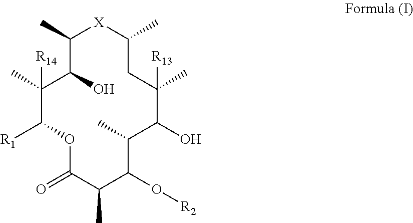

wherein X is selected from C═O, —NR₃CH₂—, —CH₂NR₃—, —NR₃(C═O)—, —(C═O)NR₃—, C═NOH, and —CH(OH)—, and R₂ is a sugar of Formula (II) or Formula (III):

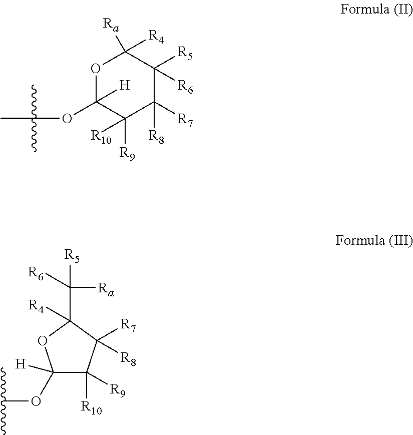

wherein R₁ is selected from an alkyl, heteroalkyl, cycloalkyl, aryl, and heteroaryl moiety, wherein alkyl moiety is selected from C₁-C₆ alkyl groups that are optionally branched, wherein heteroalkyl moiety is selected from C₁-C₆ alkyl groups that are optionally branched or substituted and that optionally comprise one or more heteroatoms, wherein cycloalkyl moiety is selected from a C₁-C₆ cyclic alkyl groups that are optionally substituted and that optionally comprise one or more heteroatoms,
wherein aryl moiety is selected from optionally substituted C₆ aromatic rings,
wherein heteroaryl moiety is selected from optionally substituted C₁-C₅ aromatic rings comprising one or more heteroatoms,
wherein heteroatoms are selected from O, N, P, and S,
wherein substituents, independently, are selected from alkyl, OH, F, Cl, NH₂, NH-alkyl, NH-acyl, S-alkyl, S-acyl, O-alkyl, and O-acyl,
wherein acyl is selected from C₁-C₄ optionally branched acyl groups,
wherein R₃ is selected from H and Me,
wherein R₄ is selected from H and Me,
wherein Rₐ is selected from H and CR₂₁R₂₂R₂₃,
wherein R₂₁, R₂₂, R₂₃, and R₅, R₆, R₇, R₈, R₉, and R₁₀, independently, are selected from H, Me, NR₁₁R₁₂, NO₂, and OR₁₁,
wherein R₂₃ together with R₄ in Formula (II), R₄ together with R₅ in Formula (II), R₅ together with R₇ in Formula (II), and R₇ together with R₉ in Formula (II), independently, may be joined to represent a bond to leave a double bond between the carbon atoms that each group is connected to,
wherein R₂₁ together with R₂₂, R₅ together with R₆, R₇ together with R₈, or R₉ together with R₁₀ may be replaced with a carbonyl,
wherein R₁₁ and R₁₂, independently, are selected from H and alkyl,
wherein R₁₃ is selected from H, OH, and OCH₃,
wherein R₁₄ is selected from H and OH,
and wherein one of R₅, R₆, R₇, R₈, R₉ or R₁₀ is selected from NR₁₁R₁₂ and NO₂,
or a pharmaceutically acceptable salt thereof.
5. The combination according to any of the preceding embodiments, wherein the macrolide is selected from:

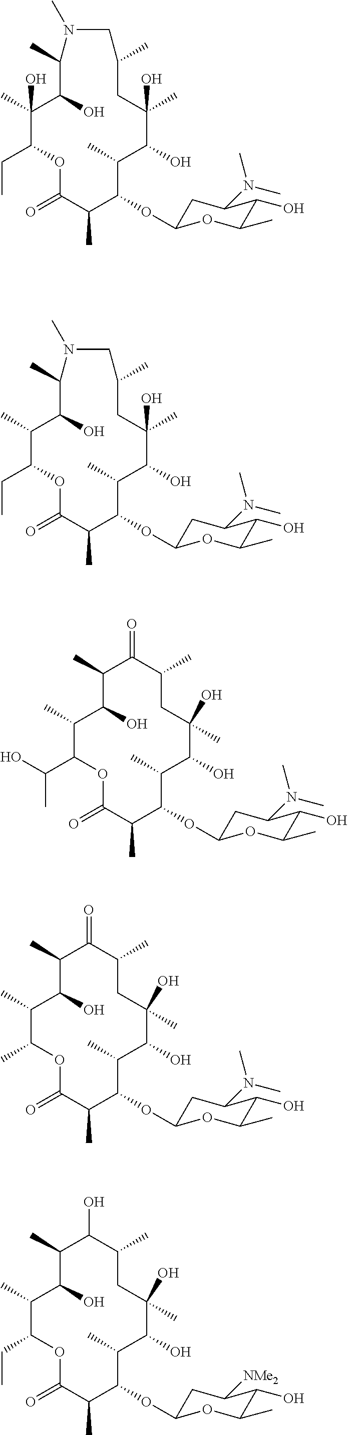

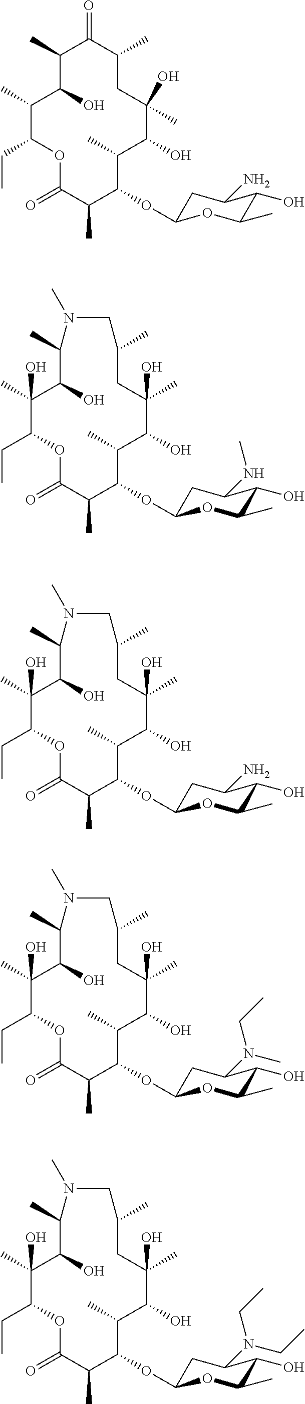

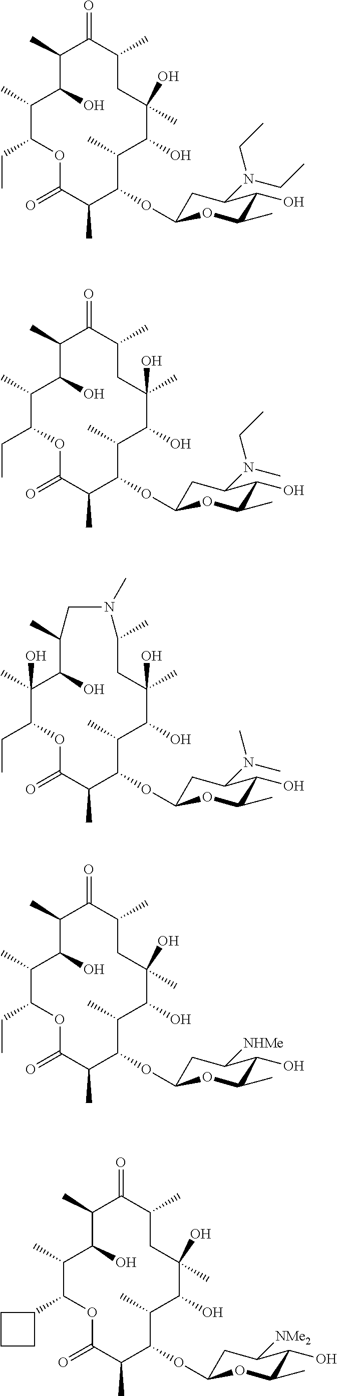

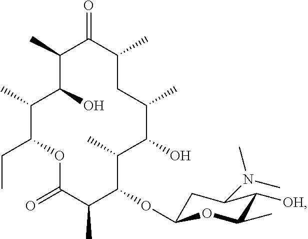

or a pharmaceutically acceptable salt thereof.
6. The combination according to any of the preceding embodiments, wherein the macrolide is

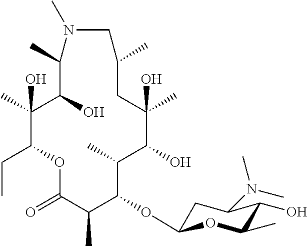

or a pharmaceutically acceptable salt thereof.
7. The combination according to any of the preceding embodiments, wherein the immune checkpoint inhibitor targets an immune checkpoint selected from cytotoxic T-lymphocyte associated antigen 4 (CTLA4, also known as CD152), programmed cell death protein 1 (PD-1, also known as CD279), PD-1 ligand 1 (PD-L1, also known as B7-H1 and CD274), PD-1 ligand 2 (PD-L2, also known as B7-DC and CD-273), T-cell membrane protein 3 (TIM3, also known as HAVcr2), adenosine A2a receptor (A2aR), lymphocyte activation gene 3 (LAG3, also known as CD 223), B7-H3 (also known as CD276), B7-H4 (also known as B7-S1, B7X, and VCTN1), 2B4 (also known as CD244), B and T lymphocyte attenuator (BTLA, also known as CD272), and CMTM6.
8. The combination according to any of the preceding embodiments, wherein the immune checkpoint inhibitor is selected from CTLA4 inhibitors, PD-1 inhibitors, PD-L1 inhibitors, PD-L2 inhibitors, TIM3 inhibitors, A2aR inhibitors, LAG3 inhibitors, B7-H3 inhibitors, B7-H4 inhibitors, 2B4 inhibitors, BTLA inhibitors, and CMTM6 inhibitors.
9. The combination according to embodiment 8 wherein the macrolide is

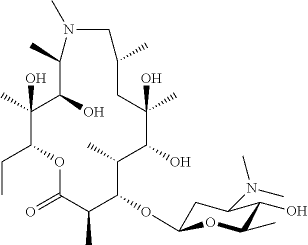

or a pharmaceutically acceptable salt thereof,
and the immune checkpoint inhibitor is a PD-1 inhibitor.
10. The combination according to any of the preceding embodiments, wherein the immune checkpoint inhibitor is selected from ipilimumab, tremelimumab, pembrolizumab, nivolumab, pidilizumab, AMP-224, atezolizumab, avelumab, durvalumab, MDX-1105, IMP321, enoblituzumab, and MGD009.
11. The combination according to embodiment 10, wherein the immune checkpoint inhibitor is selected from ipilimumab, pembrolizumab, nivolumab, atezolizumab, avelumab, and durvalumab.
12. The combination according to any of the preceding embodiments in the form of two pharmaceutical compositions, wherein one composition comprises a macrolide and one or more pharmaceutically acceptable excipients and the other composition comprises an immune checkpoint inhibitor and one or more pharmaceutically acceptable excipients.
13. The combination according to embodiment 12, wherein the two pharmaceutical compositions are designed for the same or different administration route.
14. A combination as defined in any one of embodiments 1-13 for use in medicine.
15. The combination according to embodiment 14 for use as an immunostimulator.
16. The combination according to any of embodiments 14-15 for use in the treatment of cancer.
17. The combination according to embodiment 16 wherein the cancer is selected from Adrenal Cancer, Anal Cancer, Bile Duct Cancer, Bladder Cancer, Bone Cancer, Brain/CNS Tumors, Breast Cancer, Castleman Disease, Cervical Cancer, Colon/Rectum Cancer, Endometrial Cancer, Esophagus Cancer, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Acute Myeloid Leukemia, Chronic Lymphocytic Leukemia, Acute Lymphocytic Leukemia, Chronic Myeloid Leukemia, Chronic Myelomonocytic Leukemia, Liver Cancer, Non-Small Cell Lung Cancer, Small Cell Lung Cancer, Lung Carcinoid Tumor, Lymphoma, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Basal and Squamous Cell Skin Cancer, Melanoma, Merkel Cell Skin Cancer, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor.
18. The combination according to any of embodiments 14-15 for use in the treatment of a viral disease.
19. The combination according to embodiment 18 wherein the viral disease is selected from HIV, Adenovirus, Alphavirus, Arbovirus, Borna Disease, Bunyavirus, Calicivirus, Condyloma Acuminata, Coronavirus, Coxsackievirus, Cytomegalovirus, Dengue fever virus, Contageous Ecthyma, Epstein-Barr virus, Erythema Infectiosum, Hantavirus, Viral Hemorrhagic Fever, Viral Hepatitis, Herpes Simplex Virus, Herpes Zoster virus, Infectious Mononucleosis, Influenza, Lassa Fever virus, Measles, Mumps, Molluscum Contagiosum, Paramyxovirus, Phlebotomus fever, Polyoma-virus, Rift Valley Fever, Rubella, Slow Disease Virus, Smallpox, Subacute Sclerosing Panencephalitis, Tumor Virus Infections, West Nile Virus, Yellow Fever Virus, Rabies Virus and Respiratory Syncitial Virus.
20. A pharmaceutical composition comprising a combination according to any of embodiments 1-13 and one or more pharmaceutically acceptable excipients.
21. A pharmaceutical composition comprising a combination according to any of embodiments 14-19 and one or more pharmaceutically acceptable excipients.
22. A pharmaceutical kit, comprising in a single package:
i) a first composition comprising a macrolide,
ii) a second composition comprising an immune checkpoint inhibitor, and
iii) instructions for use,
for use in treatment or prevention of cancer.
23. The pharmaceutical kit according to embodiment 22 wherein the macrolide is selected from:

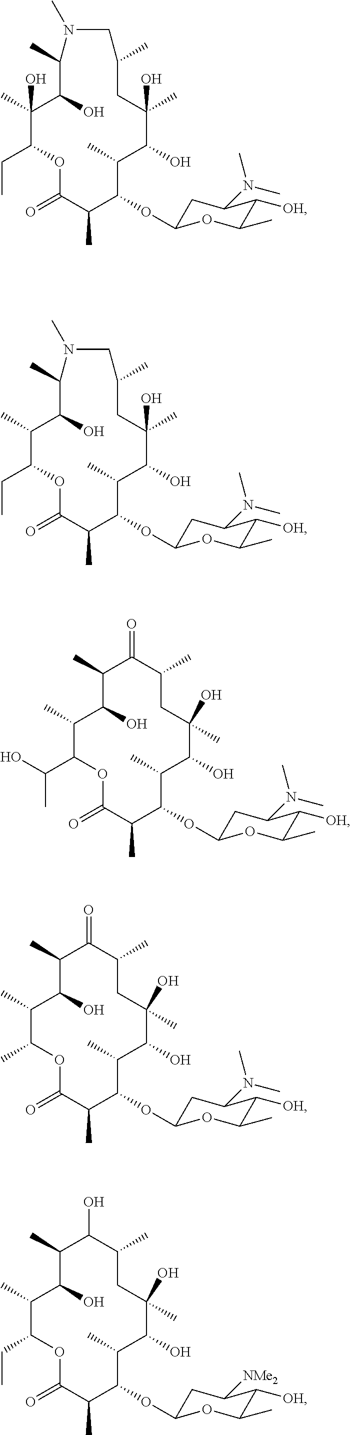

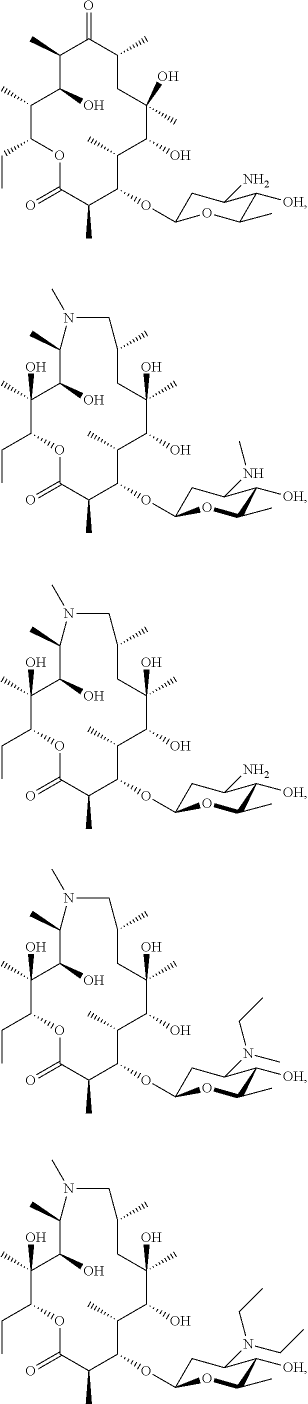

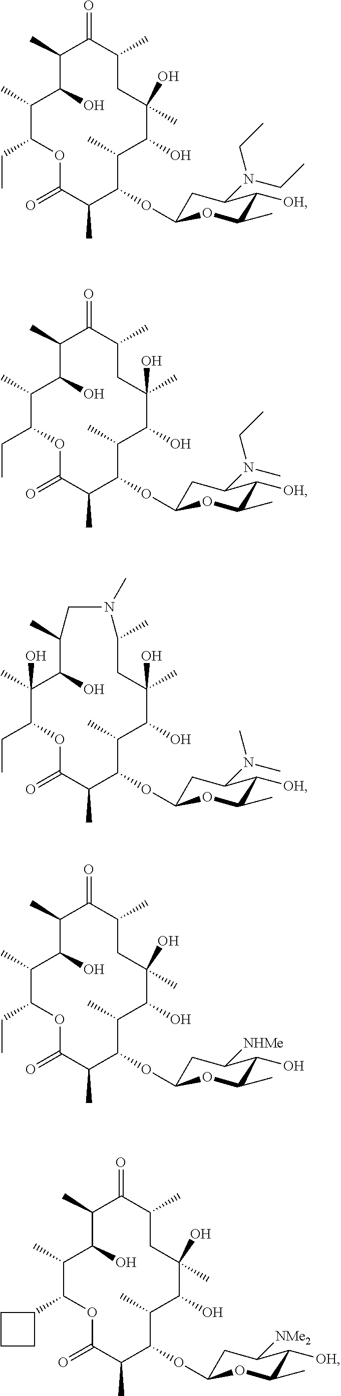

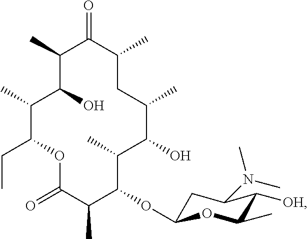

or a pharmaceutically acceptable salt thereof.
24. The pharmaceutical kit according to embodiment 21 wherein the macrolide is

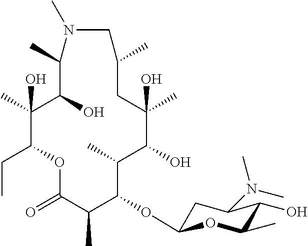

or a pharmaceutically acceptable salt thereof.
25. A method for treating or preventing cancer, the method comprising administering to a human or animal subject in need thereof a therapeutically effective amount of a combination according to any one of embodiments 1-13.

### EXPERIMENTAL

### Materials

Unless otherwise indicated, all reagents used in the examples below are obtained from commercial sources. Example suppliers of Azithromycin B include Santa Cruz Biotechnology (Texas, USA) and Toronto Research Chemicals (Toronto, Canada).

### Antibodies

Anti-CD80 V450, anti-CD69 PE, anti HLA-DR APC-R700, anti CD127-APC, and antiAnti-HLA-A,B,C FITC were purchased from BD Biosciences. Celltrace violet for T cell proliferation assay was purchased from Invitrogen. ELISA antibodies were purchased from BD Biosciences.

### Media

RPMI-1640 (Invitrogen) supplemented with 25 mM HEPES, L-glutamine, Sodium pyruvate, 10% fetal bovine serum (Gibco), 100 μg/mL penicillin and 100 μg/mL streptomycin

### General Biology Methods

The effect of the compounds of the invention on immune stimulation may be tested using one or more of the methods described below:

### General Compound Method

Compound analysis—solubility and stability in solution

### Analysis of Fermentation Broths and Compounds

An aliquot of fermentation broth obtained as described below was shaken vigorously for 30 minutes with an equal volume of ethyl acetate, and then separated by centrifugation, or the already isolated compounds were dissolved in methanol:water (9:1, 0.1 mg/ml), and then separated by centrifugation. Supernatants were analysed by LC-MS and LC-MS/MS and chromatography was achieved over base-deactivated Luna C18 reversed-phase silica (5 micron particle size) using a Luna HPLC column (250×4.6 mm; Phenomenex (Macclesfield, UK)) heated at 40° C. Agilent 1100 HPLC system comprising of quaternary pump, auto sampler, column oven and diode array detector coupled to a Bruker Esquire ion trap MS.

Mobile phase A=0.1% formic acid in water
Mobile phase B=0.1% formic acid in acetonitrile
Gradient: T=0 min, B=50%; T=4.5 min, B=50%; T=7 min, B=100%; T=10.5 min, B=100%; T=10.75 min, B=50%; T=13 min, B=50%.

Compounds were identified by LC-MS and LC-MS/MS and quantified by LC-MS/MS against an internal standard.

### Analysis of Marker Expression by Flow Cytometry

Human peripheral blood mononuclear cells (PBMCs) were purified from healthy donors with Ficoll-Paque density centrifugation. Cells were cultured in complete RPMI-1640 media (Invitrogen) supplemented with 25 mM HEPES, L-glutamine, Sodium pyruvate (Sigma), 10% fetal bovine serum, 100 μg/mL penicillin and 100 μg/mL streptomycin (Hyclone) for 24-72 hours in 37° C., 5% CO₂ and stimulated with and increasing concentrations of compound 1 and 2. Cells were then washed in PBS and stained with monoclonal antibodies specific for cell surface markers (BD Pharmingen) and analysed with flow cytomtetry using a BD FACS Canto II flow cytometer. All samples were tested in duplicates.

### Cytomegalovirus (CMV) Cultures

Human peripheral blood mononuclear cells (PBMCs) were purified from healthy CMV positive donors with Ficoll-Paque density centrifugation. The PBMC were labeled with 5 μM celltrace violet (Invitrogen) in PBS for 15 minutes and then washed with complete cell culture medium. The labeled PBMC was cultured in the presence of a peptide library spanning the CMV pp65 protein (1 μg peptide/ml, JPT) in AIM-V media (Invitrogen) supplemented with L-glutamine, Sodium pyruvate (Sigma), 10% fetal bovine serum, 100 μg/mL penicillin and 100 μg/mL streptomycin (Hyclone) for 6-8 days in 37° C., 5% CO₂. Cell proliferation was assessed with flow cytomtery using a BD FACS Canto II flow cytometer.

### ELISA

Supernatant IL-10 was measured with a standard sandwich ELISA (all antibodies from BD Biosciences) after 48 hours and 7 days incubation with 2.5 μM of compound 1 and 100 U/mL IL-2 (Miltenyi Biotechnologies) in complete RPMI media, 37° C., 5% CO₂

TLR2 Assay

Samples and controls were tested in duplicate on recombinant HEK-293-TLR cell lines using a cell reporter assay at Invivogen using their standard assay conditions. These cell lines functionally over-express human TLR2 protein as well as a reporter gene which is a secreted alkaline phosphatase (SEAP). The production of this reporter gene is driven by an NFkB inducible promoter. The TLR reporter cell lines activation results are given as optical density values (OD).

20 μl of each test article were used to stimulate the hTLR2 reporter cell lines in a 200 μl of final reaction volume. Samples were tested in duplicate, with at least two concentrations tested—20 uM and 10 uM.

### Assessment of Cell Permeability (Bidirectional)

10 μM Test article was added to the apical (A) surface of Caco-2 cell monolayers (in HBSS buffer with 0.3% DMSO and 5 μM LY at 37 degrees C.) and compound permeation into the basolateral (B) compartment measured following 90 minutes incubation. This was also performed in the reverse direction (basolateral to apical) to investigate active transport. LC-MS/MS is used to quantify levels of both the test and standard control compounds. Efflux ratio was calculated by dividing the B to A permeability by the A to B permeability.

Drug permeability:Papp=(VA/(Area×time))×([drug]accepter/(([drug]initial,donor)×Dilution Factor)

### Assessment of Metabolic Stability (Microsome Stability Assay)

Rate of metabolism in microsomes was tested as follows:

Human liver microsomes were diluted with buffer C (0.1 M Potassium Phosphate buffer, 1.0 mM EDTA, pH 7.4) to a concentration of 2.5 mg/mL. Microsomal stability studies were carried out by adding 30 μL of 1.5 μM compound spiking solution to wells (1.5 μL of 500 μM spiking solution (10 μL of 10 mM DMSO stock solution into 190 μL ACN to eventually generate final test concentration of 1 uM) and 18.75 μL of 20 mg/mL liver microsomes into 479.75 μL of Buffer C). All samples were pre-incubated for approximately 15 minutes at 37° C. Following this, the reaction was initiated by adding 15 μL of the NADPH solution (6 mM) with gentle mixing. Aliquots (40 μL) were removed at 0, 5, 15, 30 and 45 minutes and quenched with ACN containing internal standard (135 μL). Protein was removed by centrifugation (4000 rpm, 15 min) and the sample plate analysed for compound concentration by LC-MS/MS. Half-lives were then calculated by standard methods, comparing the concentration of analyte with the amount originally present.

### EXAMPLES

### Example 1—Preparation of Compound 1 (ISC397)

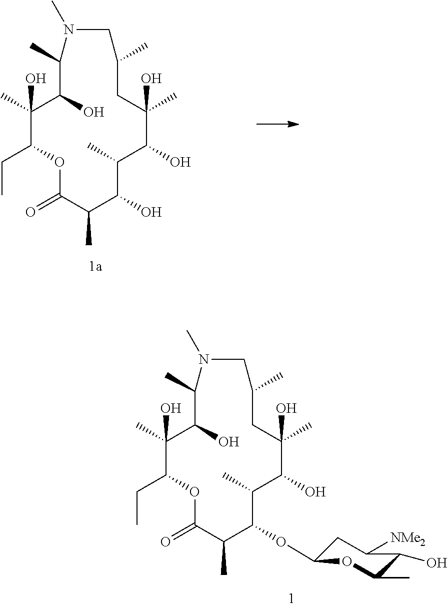

### Preparation of Azithromycin Aglycone (Az-AG) (1a)

Azithromycin aglycone (1a) was generated using methods described in the literature (Djokic et al. 1988). In brief, azithromycin is converted to azithromycin aglycone by the acidic removal of the 3-O and 5-O sugars. The 5-O amino sugar is first oxidised and pyrolyzed to facilitate cleavage.

### Generation of Biotransformation Strains Capable of Glycosylating Erythromycin Aglycones (Erythronolides):

Generation of S. erythraea 18A1 (pAES52)

pAES52, an expression plasmid containing angAI, angAII, angCVI, ang-orf14, angMIII, angB, angMI and angMII along with the actII-ORF4 pactI/III expression system (Rowe et al. 1998) was generated as follows.

The angolamycin sugar biosynthetic genes were amplified from a cosmid library of strain S. eurythermus ATCC23956 obtained from the American Type Culture Collection (Manassas, Va., USA). The biosynthetic gene cluster sequence was deposited as EU038272, EU220288 and EU232693 (Schell et al. 2008).

The biosynthetic gene cassette was assembled in the vector pSG144 as described previously (Schell et al. 2008, ESI), adding sequential genes until the 8 required for sugar biosynthesis were obtained, creating plasmid pAES52.

pAES52 was transformed into strain 18A1 (WO2005054265).

Transformation of pAES52 into S. erythraea 18A1

pAES52 was transformed by protoplast into S. erythraea 18A1 using standard methods (Kieser et al. 2000, Gaisser et al. 1997). The resulting strain was designated ISOM4522, which is deposited at the NCIMB on 24 Jan. 2017 with Accession number: NCIMB 42718.

Generation of S. erythraea SGT2 (pAES54)

pAES54, an expression plasmid containing angAI, angAII, angCVI, ang-orf14, angMIII, angB, angMI and angMII along with the actII-ORF4 pactI/III expression system (Rowe et al., 1998) was generated as follows

The angolamycin sugar biosynthetic genes were amplified from a cosmid library of strain S. eurythermus ATCC23956 obtained from the American Type Culture Collection (Manassas, Va., USA). The biosynthetic gene cluster sequence was deposited as EU038272, EU220288 and EU232693 (Schell et al. 2008)

The biosynthetic gene cassette was assembled in the vector pSG144 as described previously (Schell et al. 2008, ESI), adding sequential genes until the 8 required for sugar biosynthesis were obtained, creating plasmid pAES52.

Plasmid pAES54 was made by ligating the 11,541 bp SpeI-NheI fragment containing the actII-ORF4 pactI/III promotor system and the 8 ang genes was excised from pAES52 with the 5,087 bp XbaI-SpeI fragment from pGP9, containing an apramycin resistance gene, oriC, oriT for transfer in streptomycetes and phiBT1 integrase with attP site for integrative transformation. (The compatible NheI and XbaI sites were eliminated during the ligation.)

pAES54 was then transformed into S. erythraea SGT2 (Gaisser et al. 2000, WO2005054265).

Transformation of pAES54 into S. erythraea SGT2

pAES54 was transferred by conjugation into S. erythraea SGT2 using standard methods. In brief, E. coli ET12567 pUZ8002 was transformed with pAES54 via standard procedures and spread onto 2TY with Apramycin (50 μg/mL), Kanamycin (50 μg/mL), and Chloramphenicol (33 μg/mL) selection. This plate was incubated at 37° C. overnight. Colonies from this were used to set up fresh liquid 2TY cultures which were incubated at 37° C. until late log phase was reached. Cells were harvested, washed, mixed with spores of S. erythraea SGT2, spread onto plates of R₆ and incubated at 28° C. After 24 hours, these plates were overlaid with 1 mL of sterile water containing 3 mg apramycin and 2.5 mg nalidixic acid and incubated at 28° C. for a further 5-7 days. Exconjugants on this plate were transferred to fresh plates of R₆ containing apramycin (100 μg/mL).

### Alternative Biotransformation Strain

Alternatively, BIOT-2945 (Schell et al. 2008) may be used as the biotransformation strain, as this also adds angolosamine to erythronolides.

### Biotransformation of Azithromycin Aglycone to Prepare Compound 1

Erlenmeyer flasks (250 mL) containing SV2 medium (40 mL) and 8 uL thiostrepton (25 mg/mL) were inoculated with 0.2 mL of spore stock of strain ISOM-4522 and incubated at 30° C. and shaken at 300 rpm with a 2.5 cm throw for 48 hours.

SV2 media:

| Ingredient | Amount | |
|---|---|---|
| glycerol | 15 | g |
| glucose | 15 | g |
| soy peptone A3SC | 15 | g |
| NaCl | 3 | g |
| CaCO₃ | 1 | g |

| | |
|---|---|
| RO water | To final volume of 1 L |

Pre-sterilisation pH adjusted to pH 7.0 with 10M HCl
Sterilised by autoclaving @ 121° C., 30 minutes

Sterile bunged falcon tubes (50 mL) containing EryPP medium (7 mL) were prepared and inoculated with culture from seed flask (0.5 mL per falcon tube) without antibiotics. The falcons were incubated at 30° C. and shaken at 300 rpm with a 2.5 cm throw for 24 hours.

ERYPP medium:

| Ingredient | Amount | |
|---|---|---|
| toasted soy flour (Nutrisoy) | 30 | g |
| glucose | 50 | g |
| (NH₄)₂SO₄ | 3 | g |
| NaCl | 5 | g |
| CaCO₃ | 6 | g |

| | |
|---|---|
| RO water | To final volume of 1 L |

Pre-sterilisation pH adjusted to pH 7.0 with 10M HCl
Sterilised in situ by autoclaving @ 121° C., 30 minutes
Post sterilisation 10 ml/L propan-1-ol added

After 24 hours, azithromycin aglycone (0.5 mM in DMSO, 50 uL) was added to each falcon tube and incubation continued at 300 rpm with a 2.5 cm throw for a further 6 days.

### Isolation of Compound 1

Whole broth was adjusted to pH 9.5 and extracted twice with one volume of ethyl acetate. The organic layers were collected by aspiration following centrifugation (3,500 rpm, 25 minutes). The organic layers were combined and reduced in vacuo to reveal a brown gum that contained compound 1. This extract was partitioned between ethyl acetate (200 ml) and aqueous ammonium chloride (20 ml of a 50% concentrated solution). After separation, the organic layer was extracted with a further volume (200 ml) of the ammonium chloride aqueous solution. The combined aqueous layers were then adjusted to pH 9.0 with aqueous sodium hydroxide and then extracted twice with one volume equivalent of ethyl acetate. The organic layers were combined and reduced in vacuo to a brown solid. This extract was then applied to a silica column and eluted step wise (in 500 ml lots) with:

| Solvent | Hexanes | EtOAc | MeOH | Aq. NH₄OH |
|---|---|---|---|---|
| A | 0.499 | 0.499 | 0 | 0.002 |
| B | 0.250 | 0.748 | 0 | 0.002 |
| C | 0 | 0.998 | 0 | 0.002 |
| D | 0 | 0.988 | 0.01 | 0.002 |
| E | 0 | 0.978 | 0.02 | 0.002 |
| F | 0 | 0.968 | 0.03 | 0.002 |
| G | 0 | 0.958 | 0.04 | 0.002 |

Compound 1 was predominantly in F and G. These solvents were combined and reduced in vacuo to yield a brown solid containing compound 1. This material was then purified by preparative HPLC (C18 Gemini NX column, Phenomenex with 20 mM ammonium acetate and acetonitrile as solvent). Fraction containing the target compound were pooled and taken to dryness followed by desalting on a C₁₈ SPE cartridge.

### Example 2—Preparation of Compound 3 (Known Compound—Corresponds to Compound 17 in Schell et al., 2008)

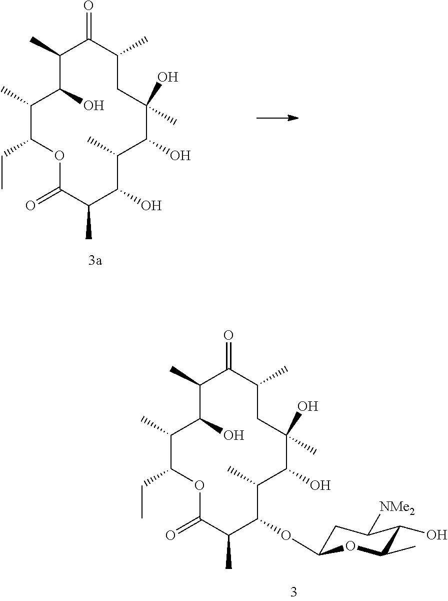

Erythronolide B (3a) can be generated by fermentation of strains of S. erythraea blocked in glycosylation, such as strains and processes described, for example, in U.S. Pat. No. 3,127,315 (e.g. NRRL2361, 2360, 2359 and 2338), Gaisser et al 2000 (e.g. S. erythraea DM ΔBV ΔCIII.

Erythronolide B (3a) was then fed to a biotransformation strain capable of adding angolosamine to the 3-hydroxyl (such as NCIMB 42718) and compound 3 was isolated from the fermentation broth by standard methods.

### Example 3—Preparation of Compound 4

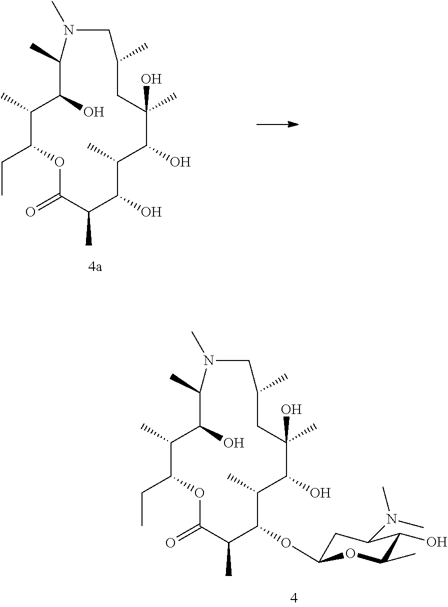

Azithromycin B aglycone (4a) was generated by hydrolysis of the sugars from azithromycin B in the same way as for azithromycin A.

Azithromycin B aglycone (4a) was then fed to a biotransformation strain capable of adding angolosamine to the 3-hydroxyl (such as NCIMB 42718) and isolated from the fermentation broth using standard methods.

### Example 4—Preparation of Compound 5

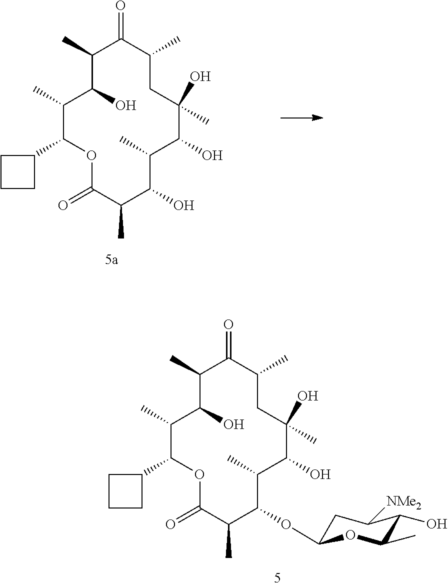

Cyclobutyl erythronolide B (5a) was generated using methods described in WO98/01571. In brief, S. erythraea DM ΔBV ΔCIII (Gaisser et al. 2000) was transformed with pIG1 (Long et al., 2002, WO98/01571). Fermentation of the resulting strain with addition of cyclobutene carboxylic acid led to production of Cyclobutyl erythronolide B (5a). This was isolated from fermentation broths using standard methods. Cyclobutyl erythronolide B (5a) was then fed to a biotransformation strain capable of adding angolosamine to the 3-hydroxyl (such as NCIMB 42718) and compound 5 isolated from the fermentation broth using standard methods.

### Example 5—Preparation of Compound 6

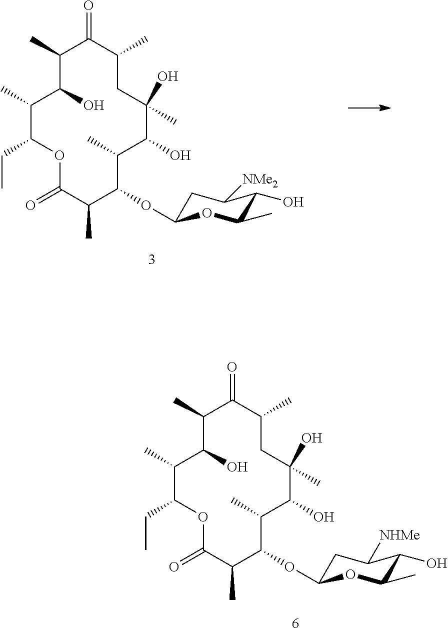

A methyl group was removed from the aminosugar of compound 3 (see example 2) by adding it to a fermentation of ATCC 31771 and isolating compound 6 from the fermentation broth using standard methods.

### Example 6—Preparation of Compound 7

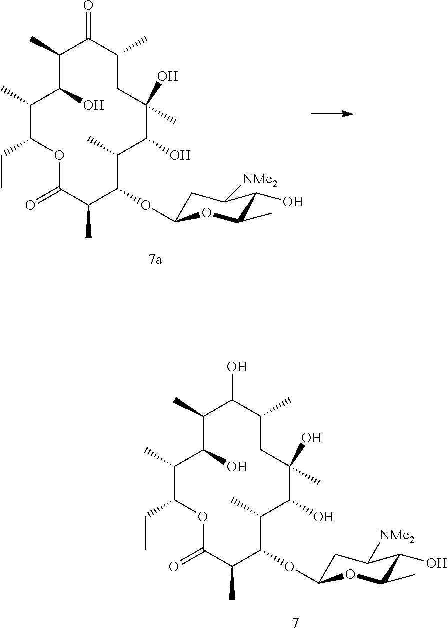

Compound 3 was treated with sodium borohydride in solvent. Following standard reaction work up compound 7 was purified by standard methods.

### Example 7—Preparation of Compound 8

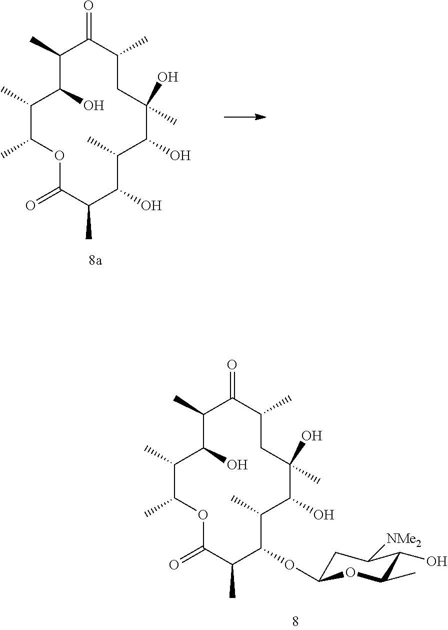

14-desmethyl erythronolide B (8a) was generated using methods described in WO2000/00618. In brief, S. erythraea DM ΔBV ΔCIII (Gaisser et al. 2000) was transformed with pPFL43. The resulting strain was fermented using typical methods and compound 8a was isolated using chromatography.

14-desmethyl erythronolide B (8a) was then fed to a biotransformation strain capable of adding angolosamine to the 3-hydroxyl (such as NCIMB 42718) and isolated from the fermentation broth using standard methods.

### Example 8—Preparation of Compound 9

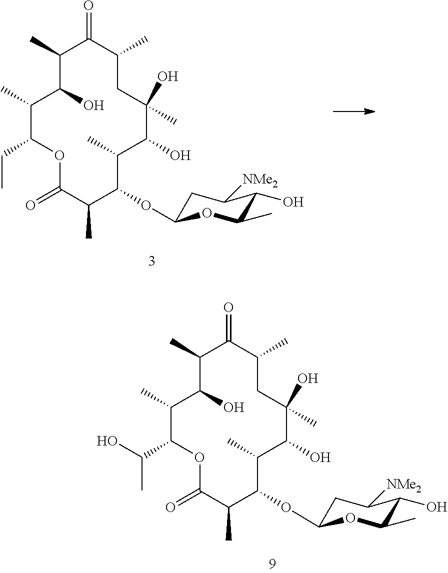

14-hydroxy angolosamine erythronolide B (9) was generated by feeding compound 3 (see example 2) to a fermentation of S. rochei ATCC 21250, which adds the hydroxyl group. Compound 9 was then isolated from the fermentation broth using standard methods.

### Example 9—Preparation of Compound 10

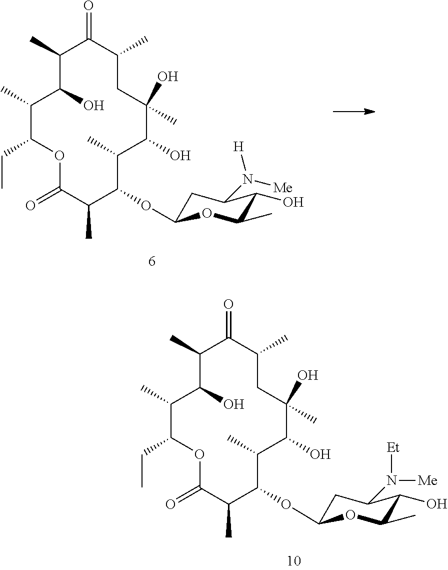

Compound 6 (6.0 mg, 0.01 mmol) was dissolved in dichloromethane (1 mL) and acetaldehyde (1.0 μL, 0.02 mmol) was added. The reaction was stirred at room temperature and sodium triacetoxyborohydride (2.1 mg, 0.01 mmol) was added. The reaction was stirred for 30 minutes and then quenched by the addition of concentrated aqueous sodium bicarbonate (25 mL). The aqueous extract was extracted with ethyl acetate (3×25 mL). The organic extracts were combined, washed with concentrated brine solution and the solvent was removed in vacuo. The target compound 10 was then purified by preparative HPLC.

### Example 10—Preparation of Compound 12

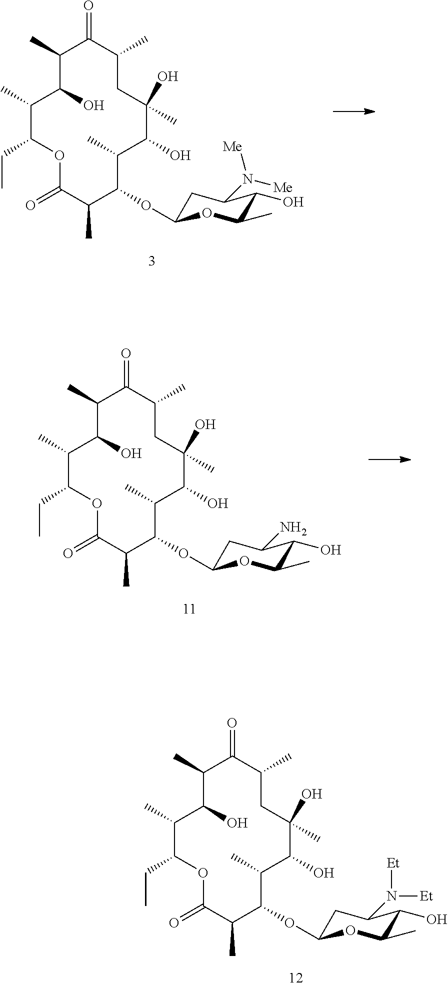

Compound 3 (see example 2) was biotransformed to remove both methyl groups from the aminosugar by adding it to a fermentation of ATCC 31771 and compound 11 was isolated from the fermentation broth using standard methods.

Compound 11 is dissolved in THF and acetaldehyde is added. The reaction is stirred at room temperature and sodium cyanoborohydride is added. The reaction is stirred further and the reaction is quenched by the addition of aqueous sodium bicarbonate. The aqueous extract is extracted with EtOAc (3× vol equivalent). The organic extracts are combined, washed with brine and the solvent is removed in vacuo. The target compound 12 is then purified using standard methods.

### Example 11—Preparation of Compound 14

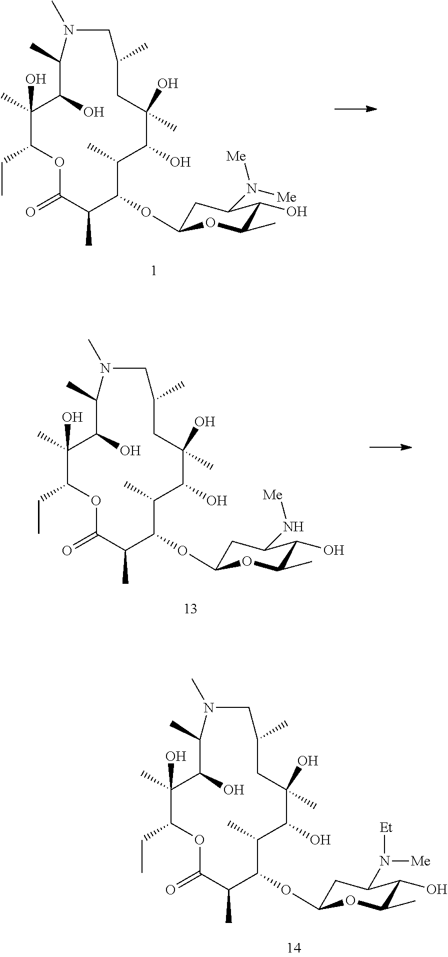

Compound 1 (see example 1) is biotransformed to remove a methyl group from the aminosugar by adding it to a fermentation of ATCC 31771 and compound 13 is isolated from the fermentation broth using standard methods.

Compound 13 is dissolved in THF and acetaldehyde is added. The reaction is stirred at room temperature and sodium cyanoborohydride is added. The reaction is stirred further and the reaction is quenched by the addition of aqueous sodium bicarbonate. The aqueous extract is extracted with EtOAc (3×vol equivalent). The organic extracts are combined, washed with brine and the solvent is removed in vacuo. The target compound 14 is then purified using standard methods.

### Example 12—Preparation of Compound 16

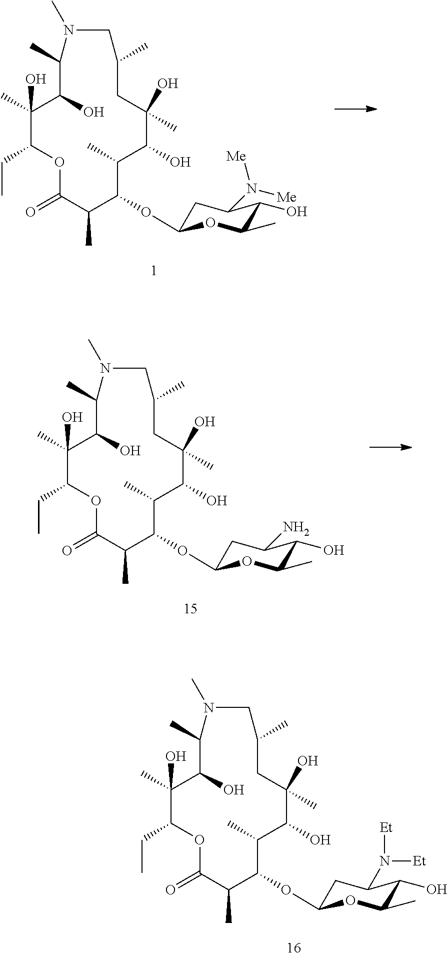

Compound 1 (see example 1) is biotransformed to remove both methyl groups from the aminosugar by adding it to a fermentation of ATCC 31771 and compound 15 is isolated from the fermentation broth using standard methods.

Compound 15 is dissolved in THF and acetaldehyde is added. The reaction is stirred at room temperature and sodium cyanoborohydride is added. The reaction is stirred further and the reaction is quenched by the addition of aqueous sodium bicarbonate. The aqueous extract is extracted with EtOAc (3×vol equivalent). The organic extracts are combined, washed with brine and the solvent is removed in vacuo. The target compound 16 is then purified using standard methods.

### Example 13—Assessment of Direct Antibacterial Activity

The bioactivity of macrolide compounds against 4 strains of common gut bacteria (Escherichia coli, Streptococcus salivarius subsp. salivarius, Lactobacillus casei and Bifidobacterium longum subsp. infantis) and common mammalian skin isolate Micrococcus luteus, was assessed using the Minimum Inhibitory Concentration (MIC) assay. Bacterial strains were purchased from DSMZ (Brunswick, Germany) except M. luteus which was obtained from NCIMB, and stored in 20% glycerol at −80° C.

Stock solutions (100% DMSO) of positive controls (azithromycin and erythromycin), and of test compounds 1 and 2 were diluted in broth to working stock concentrations of 256 μg/ml (final assay testing concentration range 128 μg/ml to 0.00391 μg/ml). Stock solutions of all other compounds were diluted in broth to working stock concentrations of 128 μg/ml (final assay testing concentration range 64 μg/ml to 0.00195 μg/ml).

Bacterial strains were cultivated in appropriate broth in an anaerobic chamber at 37° C., except for M. luteus which was incubated aerobically at 37° C. 18 h cultures were diluted in broth to an OD₅₉₅ of 0.1 and then further diluted 1:10. In 96-well plates, in duplicate, 200 μl working stock of test compound was transferred to well 1 and serially diluted (1:2) in broth. 100 μl bacterial suspension was aliquoted into each well and mixed thoroughly. Appropriate sterility controls were included and plates were incubated in an anaerobic chamber, or aerobically (M. luteus) at 37° C. for 18 h. The MIC was determined to be the concentration of test compound in the first well with no visible growth.

TABLE 1

| | Escherichia | Streptococcus | | Lactobacillus | | Bifidobacterium | Micrococcus | |
|---|---|---|---|---|---|---|---|---|
| | coli | salivarius | | casei | | longum | luteus | |
| Azithromycin | <8 μg/ml | <0.5 | μg/ml | <1.0 | μg/ml | >64 μg/ml | 0.125 | μg/ml |
| Erythromycin | >64 μg/ml | <0.06 | μg/ml | <0.25 | μg/ml | >64 μg/ml | <0.0625 | μg/ml |
| Compound 1 | >64 μg/ml | >64 | μg/ml | >64 | μg/ml | >64 μg/ml | >256 | μg/ml |
| Compound 4 | >64 μg/ml | >64 | μg/ml | >64 | μg/ml | >64 μg/ml | | |
| Compound 5 | >64 μg/ml | >64 | μg/ml | >64 | μg/ml | >64 μg/ml | | |
| Compound 6 | >64 μg/ml | >64 | μg/ml | >64 | μg/ml | >64 μg/ml | | |
| Compound 7 | >64 μg/ml | >64 | μg/ml | >64 | μg/ml | >64 μg/ml | | |
| Compound 8 | >64 μg/ml | >64 | μg/ml | >64 | μg/ml | >64 μg/ml | | |
| Compound 9 | >64 μg/ml | >64 | μg/ml | >64 | μg/ml | >64 μg/ml | | |
| EM703 | | | | | | | 64-128 | μg/ml |

As can be seen from the data presented in Table 1, compounds 1, 3, 4, 5, 6, 7, 8 and 9 show no antibacterial activity against any of the bacterial strains tested, whilst erythromycin and azithromycin show potent activity against a number of the strains.

### Example 14—Assessment of Immune Stimulatory Activity

Human peripheral blood mononuclear cells (PBMCs) were purified from healthy donors with Ficoll-Paque density centrifugation. Cells were cultured in complete RPMI-1640 medium (Invitrogen) supplemented with 25 mM HEPES, L-glutamine, Sodium pyruvate (Sigma), 10% fetal bovine serum, 100 μg/mL penicillin and 100 μg/mL streptomycin (Hyclone). Cells were stimulated for 24 h (study 1-4) or 48 h to 1 week (study 5) in 37° C., 5% CO₂ with increasing concentrations of compound 1 and 2 in tissue culture plates. The cells were removed from the plate, washed in PBS and analysed for expression of cell specific surface markers and MHC class I with flow cytomtery using monoclonal antibodies from BD Pharmingen and a FACS Canto II flow cytometer.

Supernatant IL-10 was measured with a standard sandwich ELISA (all antibodies from BD Biosciences) after 48 hours and 7 days incubation with 2.5 uM of compound 1 and 100 U/mL IL-2 (Miltenyi Biotechnologies) in complete RPMI media, 37° C., 5% CO₂

Study 1: After 24 h of in vitro stimulation of peripheral blood mononuclear cells (PBMC) with 1 μM compound 1 (FIG. 8) the activation marker CD69 was upregulated on CD4+ T cells and B cells (FIG. 1).

Study 2: We also observed upregulation of the molecule MHC class I (HLA-ABC) on T- and B-cells (FIG. 2), indicating an effect on antigen presentation of viral antigens.

Study 3: Stimulation of PBMC with compound 1 led to the upregulation of the co-stimulatory molecule CD80 as well as the antigen presenting molecule MHC class II (HLA-DR) on monocytes (FIG. 3).

Study 4: Monocytes differentiated into macrophages also upregulated CD80 in response to stimulation by compound 1 (FIG. 4).

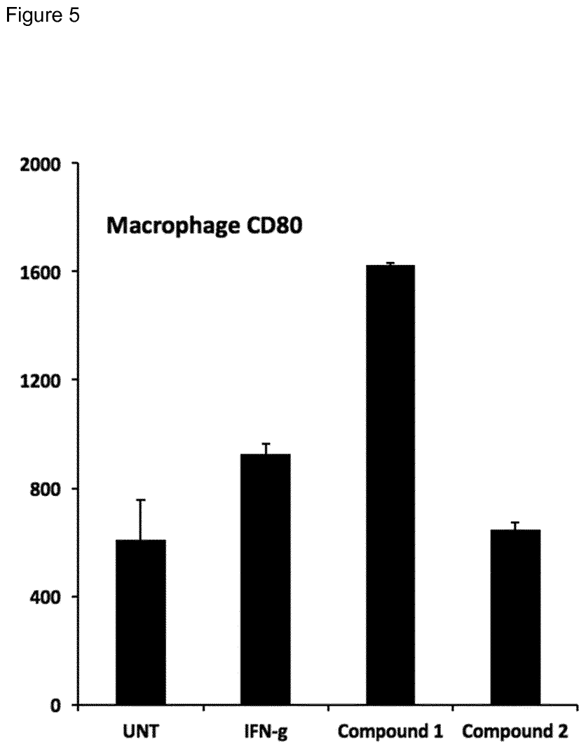

Study 5: PBMCs stimulated with compound 1 for 48 h and 7 days expressed an altered cytokine profile with increased production of the immunosuppressive cytokine IL-10, measured with sandwich ELISA. This indicate an immune inhibitory effect under certain conditions (FIG. 5).

Study 6: PBMC were stimulated with compound 1 and cultured in RPMI media for 6 days in the presence of IL-2 (Miltenyi Biotechnologies) and Cell Trace Violet Dye (Invitrogen). Proliferation was measured with flow cytometry. Analysis of the immunological effect of compound 1 revealed an altered cytokine driven proliferation profile of T cells (FIG. 6).

Study 7: Virus specific T cell proliferation was also affected by compound 1. PBMCs from cytomegalovirus (CMV) infected donors cultured in the presence of CMV antigen and compound 1 for 6 days displayed an altered phenotype of activated CMV specific CD8+ T cells with an increased expression of IL-7 receptor α (CD127), measured with flow cytometry (FIG. 7). CD127 is crucial for T cell homeostasis, differentiation and function, and reduced expression correlates with disease severity in HIV and other chronic viral diseases (Crawley et al. 2012).

As can be seen, compound 1 has a surprising ability to specifically activate and modify an immune response by affecting antigen presentation, co-stimulation and T cell activation and proliferation. In many of these studies, compound 2, another related macrolide erythromycin analogue with altered glycosylation, previously published in Schell et al, 2008 (as compound 20), was included and showed little or no activity in the assays.

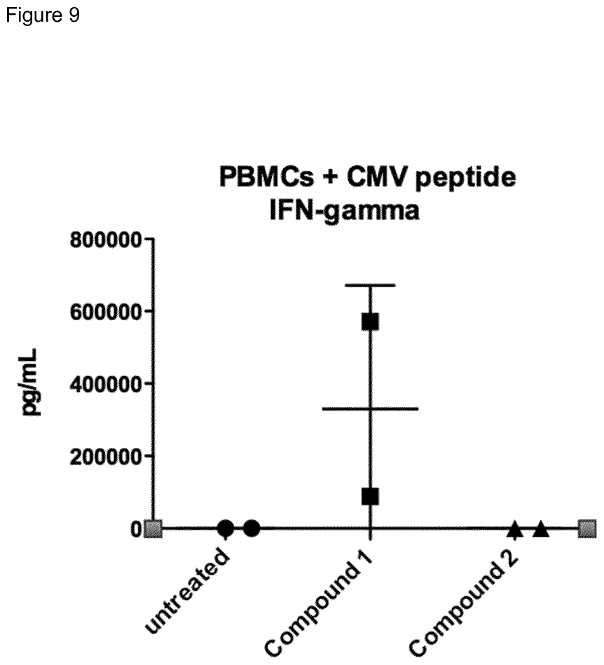

Study 8: PBMCs from CMV infected donors cultured in the presence of CMV antigen where either untreated or exposed to compound 1 or compound 2 for 3 days. Exposure to compound 1 induced secretion of high levels of IFN-gamma, whereas antigen culture alone or antigen together with compounds A did not induce IFN-gamma secretion (FIG. 9).

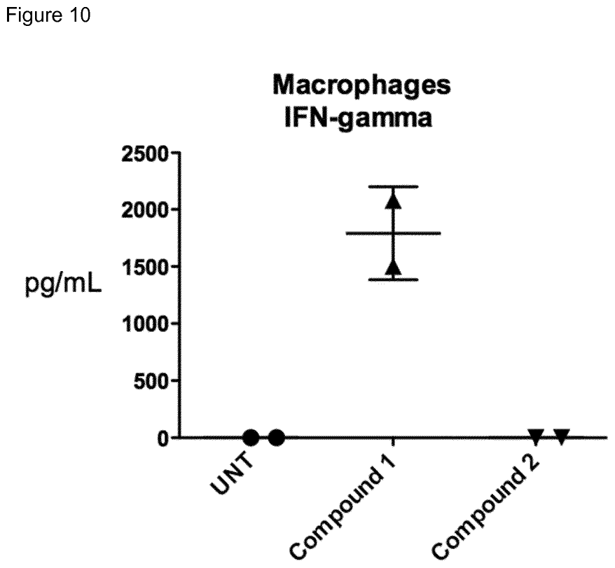

Study 9: Macrophages from healthy donors where exposed to compounds 1 or 2 for 48 hours. Only macrophages exposed to compound 1 secreted IFN-gamma whereas untreated macrophages and macrophages exposed to compound A did not secrete IFN-gamma (FIG. 10). Compound 1 is therefore able to induce IFN-gamma secretion in macrophages from healthy donors.

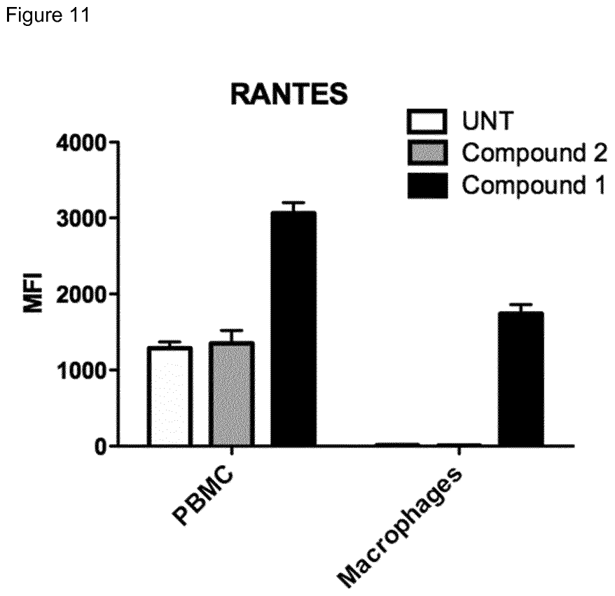

Study 10: PBMCs and macrophages where exposed to compounds 1 or 2 for 2 days (FIG. 11). Basal expression of RANTES in PBMCs was unaffected by compound 2, whereas compound 1 induced a twofold upregulation of expression. Expression of RANTES was miniscule in macrophages, and compound 1 induced a high expression.

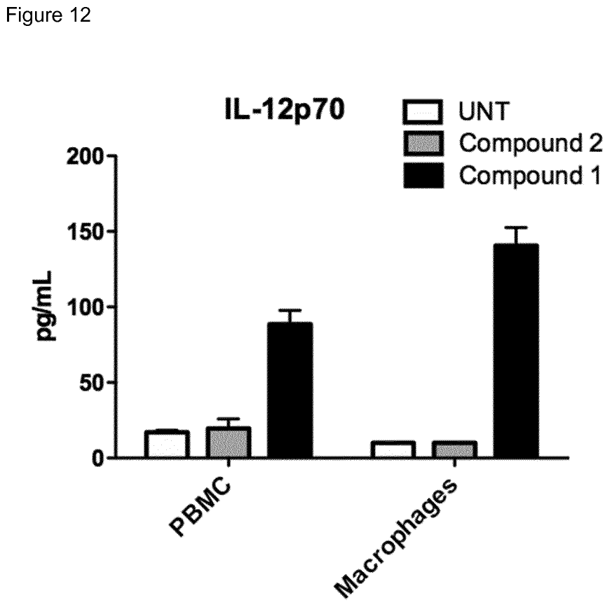

Study 11: PBMCs and macrophages where exposed to compounds 1 and 2 for 2 days. PBMCs and macrophages secreted IL-12p70 in response to compound 1, whereas compound 2 failed to induce secretion over untreated cells (FIG. 12).

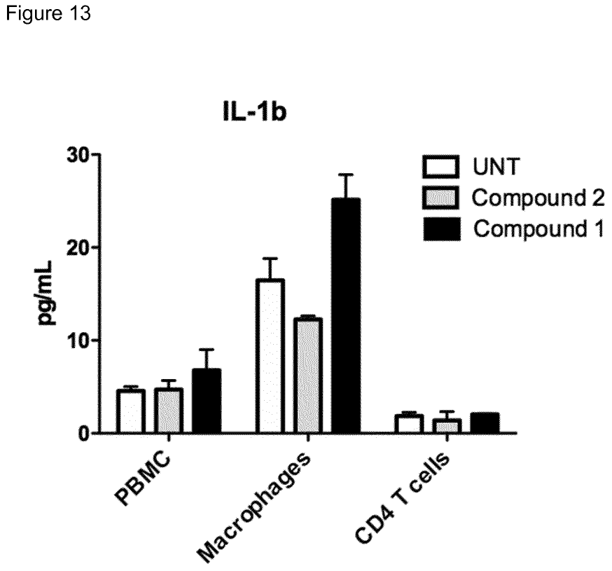

Study 12: PBMCs, macrophages and CD4+ T cells where exposed to compounds 1 and 2 for 2 days. IL-1 beta secretion was increased by compound 1 in macrophages and slightly in PBMCs while no IL-1 beta was induced in CD4+ T cells (FIG. 13).

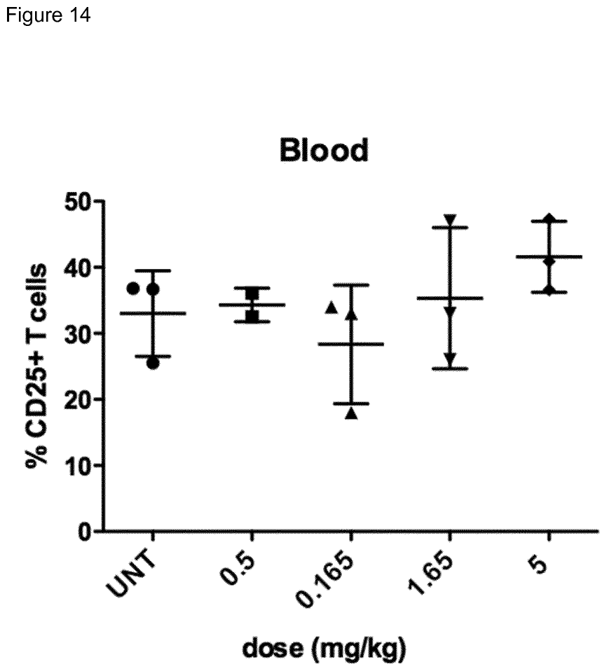

Study 13: Compound 1 was administered i.v. to C57bl/6 mice at 0.165 mg/kg to 5 mg/kg. CD25+ cell abundance was increased in animals receiving the highest dose of 5 mg/kg (FIG. 14), as was body weight in the same group (not shown).

Study 14: Compound 1 or 2 was administered i.v. to C57bl/6 mice. 24 h later the spleen was removed and MHC class I expression on CD11 b+ splenocytes was assessed Compound 1 induced an increase in splenocyte cells with high MHC I expression, whereas no effect was observed in splenocytes from mice injected with compound A.

### Example 15—Assessment of Metabolic Stability

The metabolic stability of the compounds of the invention was assessed in a standard human microsome stability assay (see general methods). Compounds with longer half-lives would be expected to have longer half-lives following dosing, which can be useful to allow less frequent dosing. Compounds with shorter half-lives could be useful for use as ‘soft drugs’ where the active entity degrades rapidly once entering the patient's system. The half-life of the compounds assessed in shown in table 2 below:

TABLE 2

| | T½ (minutes) |
|---|---|
| Azithromycin | 245 |
| Erythromycin | 31 |
| Compound 1 | 108 |
| Compound 3 | 35 |
| Compound 4 | 160 |
| Compound 5 | 83 |
| Compound 6 | 109 |
| Compound 7 | 56 |
| Compound 8 | 33 |
| Compound 9 | 100 |
| Compound 10 | 31 |
| Compound 17 | 151 |
| Compound 18 | 25 |
| Compound 19 | 18 |
| EM703 | 97 |

As can be seen, many of the compounds of the invention have increased or decreased metabolic stability as compared to azithromycin, erythromycin and EM703 (e.g. see EP1350510).

### Example 16—Assessment of Caco-2 Permeability

The permeability of the compounds of the invention was assessed in a standard caco-2 bidirectional permeability assay (see general methods). Compounds with increased permeability would be expected to have better cell penetration and potential for effect, those with improved permeability and/or reduced efflux would be expected to have increased oral bioavailability. The permeability and efflux of the compounds is shown in table 3 below:

TABLE 3

| | Pₐₚₚ × 10⁶/cm · s⁻¹ | Efflux ratio |
|---|---|---|
| Azithromycin | <0.14 | >78 |
| Compound 1 | 0.32 | 63 |
| Compound 3 | 0.27 | 166 |
| Compound 4 | 0.38 | 49 |
| Compound 5 | 0.47 | 81 |
| Compound 8 | 0.46 | 56 |
| Compound 10 | 1.23 | 26 |
| Compound 17 | 0.5 | 39 |
| Compound 18 | 9.44 | 3.5 |
| EM703 | <0.15 | >108 |

As can be seen, many of the compounds of the invention have improved cell permeability and/or reduced efflux as compared to azithromycin and EM703 (e.g. see EP1350510).

### Example 17—Assessment of TLR2 Stimulation

Compounds were tested using a TLR2 reporter assay (see general methods) that measured for stimulation of the TLR2 receptor. Stimulatory effect was measured as an increase in optical density (OD) due to release of secreted alkaline phosphatase (SEAP) and is shown in table 4:

TABLE 4

| | OD after | OD after | OD after |
|---|---|---|---|
| | addition of | addition of | addition of |
| | 20 μM | 10 μM | 5 μM |
| | test article | test article | test article |
| Azithromycin | 0.031 | 0.045 | 0.029 |
| Erythromycin | 0.045 | 0.065 | 0.035 |
| Compound 1 | 0.458 | 0.202 | 0.111 |
| Compound 2 | 0.044 | 0.010 | 0.046 |
| Compound 3 | −0.026 | −0.015 | −0.043 |
| Compound 17 | 0.234 | 0.155 | 0.054 |
| EM703 | −0.033 | −0.024 | −0.040 |

As can be seen, compound 1 stimulated TLR2 at concentrations down to 5 uM, compound 17 stimulated TLR2 at concentrations down to 10 uM, whilst erythromycin A, azithromycin and compounds 2 and 3, related macrolide erythromycin analogues with altered glycosylation, previously published in Schell et al, 2008 (as compounds 17 and 20), showed little or no stimulation at concentrations up to 20 uM.

### Example 18—Preparation of Compound 17

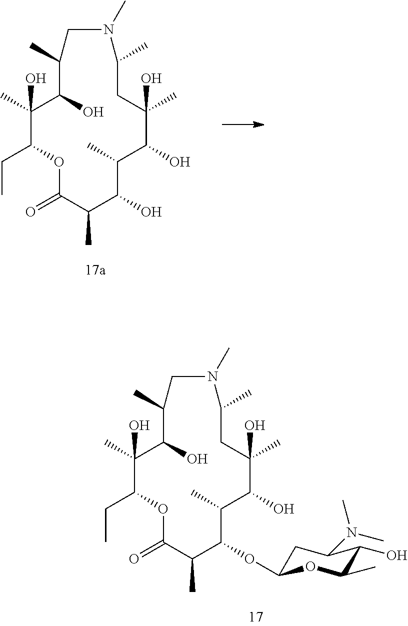

The aglycone 17a was generated from 9-deoxo-8a-aza-8a-methyl-8a-homoerythromycin (Wilkening 1993) followed by hydrolysis of the sugars. 17a was then fed to a biotransformation strain capable of adding angolosamine to the 3-hydroxyl (such as NCIMB 42718) and compound 17 isolated from the fermentation broth using standard methods.

### Example 19—Preparation of Compound 18

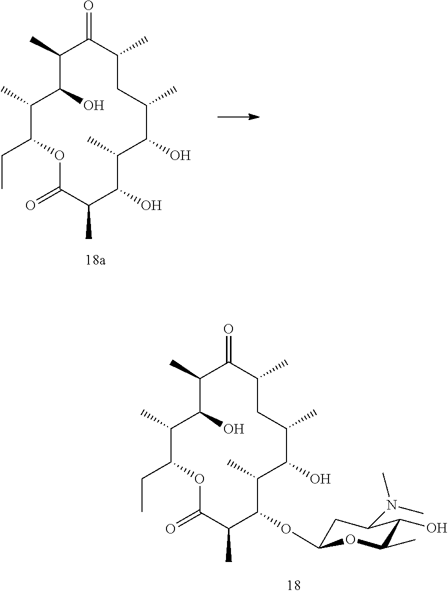

6-deoxy erythronolide B (6-DEB, 18a) was fed to a biotransformation strain capable of adding angolosamine to the 3-hydroxyl (such as NCIMB 42718) and isolated from the fermentation broth using standard methods.

### Example 20—Study of Effect of Combination of ISC397 and Checkpoint Inhibitor

C57BL/6J mice were purchased from Charles River Laboratories, Germany. The mice were injected subcutaneously into the right rear flank with 1×106 B16-F10 melanoma cells under isoflurane anesthesia.

The treatment groups were (10 mice per group):
1) Anti-PD-1 (clone RMP 1-14 from Merck, Johnson, Roche or Astra, 200 microgram/dose) on day 1, 3, 6, 9 and 12.
2) Anti-PD-1 (clone RMP 1-14, from Merck, Johnson, Roche or Astra, 200 microgram/dose) on day 1, 3, 6, 9 and 12+ISR397 (500 microgram/dose) daily until termination of the experiment.
3) Untreated.

All compounds were injected i.v. The tumor volume was measured daily and the health status of the animals twice daily. Animals were killed if the tumors reached 2 ml or if the health status were poor. The experiment was terminated on day 18 and all mice killed by cervical dislocation.

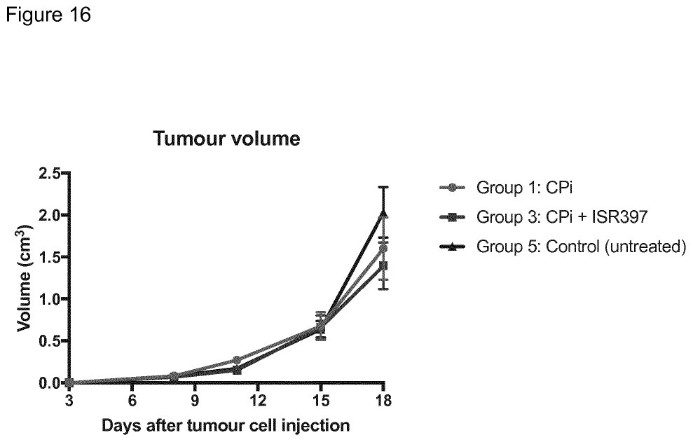

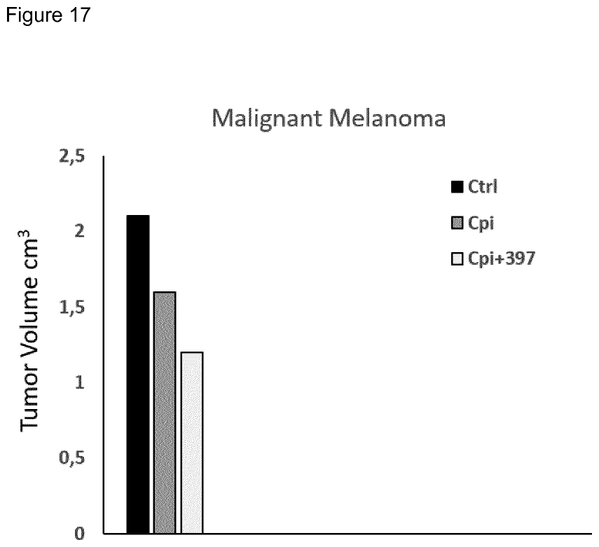

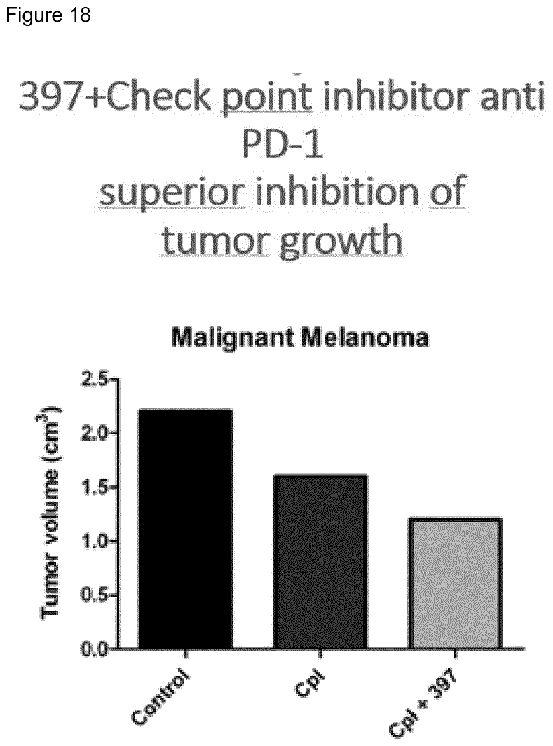

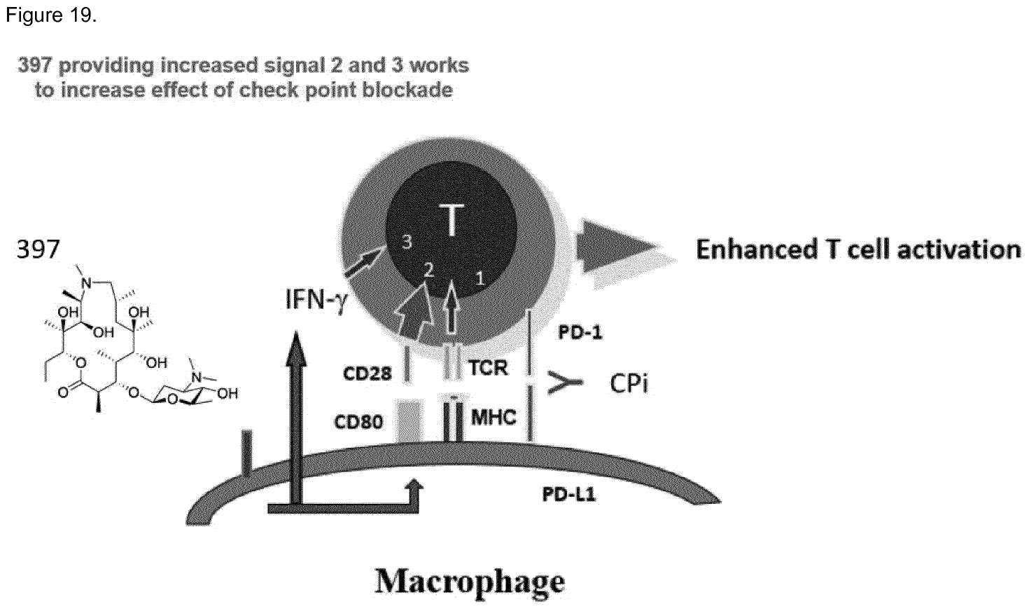

The results are shown in FIGS. 16-19.

### REFERENCES

Kieser et al. 2000 Practical Streptomyces Genetics, Published by the John Innes Foundation
Crawley et al. The influence of HIV on CD127 expression and its potential implications for IL-7 therapy. Semin Immunol. 2012 June; 24(3):231-40.
Gaisser et al. Analysis of seven genes from the eryAl-eryK region of the erythromycin biosynthetic gene cluster in Saccharopolyspora erythraea. Mol. Gen. Genet., 1997 October; 256(3):239-51.
Gaisser et al. A defined system for hybrid macrolide biosynthesis in Saccharopolyspora erythraea Mol. Micro., 2000; 36(2):391-401
Schell et al. Engineered biosynthesis of hybrid macrolide polyketides containing D-angolosamine and D-mycaminose moieties Org. Biomol. Chem., 2008; 6:3315-3327
LeMahieu et al. Glycosidic Cleavage Reactions on Erythromycin A. Preparation of Erythronolide A, J. Med. Chem., 1974, 17(9):953-956
Djokic et al. Erythromycin Series. Part 13. Synthesis and Structure Elucidation of 10-Dihydro-10-deoxo-11-methyl-11-azaerythromycin A J. Chem. Res. (S), 1988; 5:152-153
Glansdorp et al. Using Chemical Probes to Investigate the Sub-Inhibitory Effects of Azithromycin, Org. Biolmol. Chem., 2008; 208(6): 4120-4124
Rowe et al. Construction of new vectors for high-level expression in actinomycetes. Gene. 1998 Aug. 17; 216(1):215-23.
Long et al. Engineering specificity of starter unit selection by the erythromycin-producing polyketide synthase. Mol. Microbiol. 2002 March; 43(5):1215-25.
Wilkening et al. The synthesis of novel 8a-aza-8a-homoerythromycin derivatives via the Beckmann rearrangement of (9Z)-erythromycin A oxime, Bioorg. Med. Chem Lett. 1993, 3 (6), p 1287-1292

All references referred to in this application, including patent and patent applications, are incorporated herein by reference to the fullest extent possible.

1. A combination comprising a macrolide and an immune checkpoint inhibitor, wherein the macrolide has the structure of Formula (I): 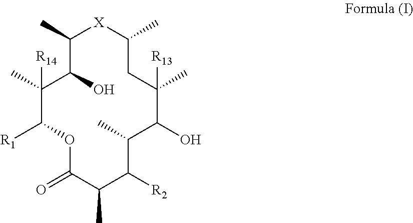
or a pharmaceutically acceptable salt thereof, wherein:
X is selected from —NR₃CH₂—, —CH₂NR₃—, —NR₃(C═O)—, —(C═O)NR₃—, and C═NOH;
R₂ is a sugar of Formula (II) or Formula (III): 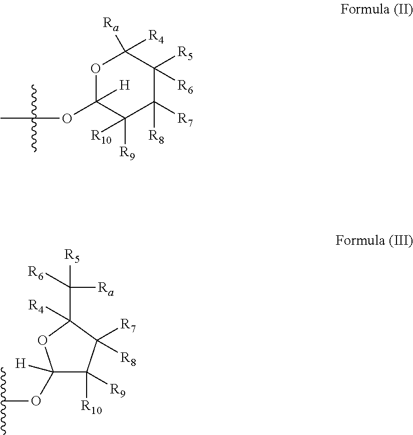
R₁ is selected from an alkyl, heteroalkyl, cycloalkyl, aryl, and heteroaryl moiety; wherein
the alkyl moiety is selected from C₁-C₆ alkyl groups that are optionally branched;
the heteroalkyl moiety is selected from C₁-C₆ alkyl groups that are optionally branched or substituted and that comprise one or more heteroatoms;
the cycloalkyl moiety is selected from C₃-C₆ cyclic alkyl groups that are optionally substituted and that optionally comprise one or more heteroatoms;
the aryl moiety is selected from optionally substituted C₆ aromatic rings;
the heteroaryl moiety is selected from optionally substituted C₁-C₅ aromatic rings comprising one or more heteroatoms;
the heteroatoms are selected from O, N, P, and S;
the substituents, independently, are selected from alkyl, OH, F, Cl, NH₂, NH-alkyl, NH-acyl, S-alkyl, S-acyl, O-alkyl, and O-acyl; and
acyl is selected from C₁-C₄ optionally branched acyl group;
R₃ is selected from H and Me;
R₄ is selected from H and Me;
Rₐ is selected from H and CR₂₁R₂₂R₂₃;
R₂₁, R₂₂, R₂₃, and R₅, R₆, R₇, R₈, R₉, and R₁₀, independently, are selected from H, Me, NR₁₁R₁₂, NO₂, and OR₁₁;
R₂₃ together with R₄ in Formula (II), R₄ together with R₅ in Formula (II), R₅ together with R₇ in Formula (II), and R₇ together with R₉ in Formula (II), independently, may be joined to represent a bond to form a double bond between the carbon atoms that each group is connected to;
R₂₁ together with R₂₂, R₅ together with R₆, R₇ together with R₈, or R₉ together with R₁₀ may form a carbonyl;
R₁₁ and R₁₂, independently, are selected from H and alkyl;
R₁₃ is selected from H, OH, and OCH₃;
R₁₄ is selected from H and OH; and
one of R₅, R₆, R₇, R₈, R₉ or R₁₀ is selected from NR₁₁R₁₂ and NO₂.

2. The combination according to claim 1, wherein the macrolide is selected from: 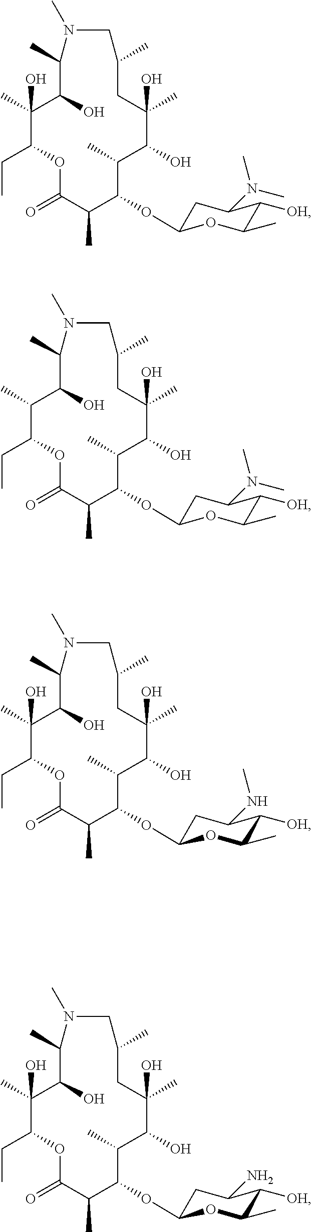 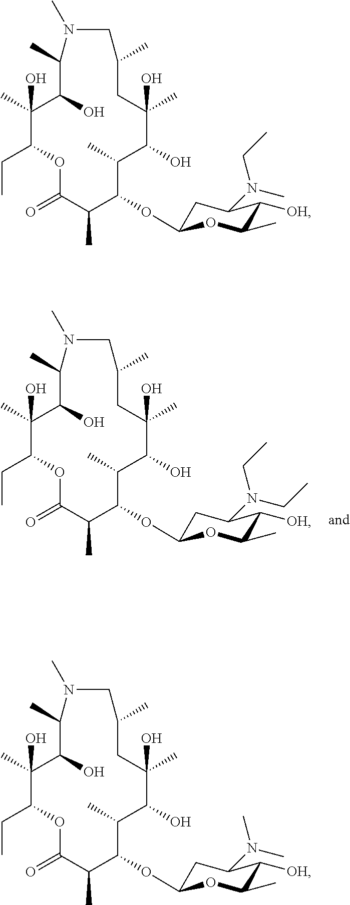
or a pharmaceutically acceptable salt thereof.

3. The combination according to claim 1, wherein the macrolide is: 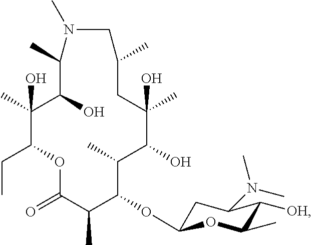
or a pharmaceutically acceptable salt thereof.

4. The combination according to claim 1, wherein the immune checkpoint inhibitor targets an immune checkpoint selected from cytotoxic T-lymphocyte associated antigen 4 (CTLA4), programmed cell death protein 1 (PD-1), PD-1 ligand 1 (PD-L1), PD-1 ligand 2 (PD-L2), T-cell membrane protein 3 (TIM3), adenosine A2a receptor (A2aR), lymphocyte activation gene 3 (LAG3), B7-H3, B7-H4, 2B4, B and T lymphocyte attenuator (BTLA), and CMTM6.

5. The combination according to claim 1, wherein the immune checkpoint inhibitor is selected from CTLA4 inhibitors, PD-1 inhibitors, PD-L1 inhibitors, PD-L2 inhibitors, TIM3 inhibitors, A2aR inhibitors, LAG3 inhibitors, B7-H3 inhibitors, B7-H4 inhibitors, 2B4 inhibitors, BTLA inhibitors, and CMTM6 inhibitors.

6. The combination according to claim 1, wherein the immune checkpoint inhibitor is selected from ipilimumab, tremelimumab, pembrolizumab, nivolumab, pidilizumab, AMP-224, atezolizumab, avelumab, durvalumab, MDX-1105, IMP321, enoblituzumab, and MGD009.

7. The combination according to claim 1, wherein the immune checkpoint inhibitor is selected from ipilimumab, pembrolizumab, nivolumab, atezolizumab, avelumab, and durvalumab.

8. The combination according to claim 1, wherein the combination comprises a first pharmaceutical composition and a second pharmaceutical composition, the first pharmaceutical composition comprising the macrolide and one or more pharmaceutically acceptable excipients, and the second pharmaceutical composition comprising the immune checkpoint inhibitor and one or more pharmaceutically acceptable excipients.

9. The combination according to claim 8, wherein the first and second pharmaceutical compositions are designed for the same administration route.

10. A method of activating or stimulating the immune system in a subject in need thereof, comprising administering to the subject an effective amount of the combination according to claim 1.

11. A method of treating cancer in a subject in need thereof, comprising administering to the subject the combination according to claim 1.

12. A method of treating a viral disease in a subject in need thereof, comprising administering to the subject the combination according to claim 1.

13. A pharmaceutical composition comprising the combination according to claim 1 and one or more pharmaceutically acceptable excipients.

14. A method of treating cancer in a subject in need thereof, comprising administering to the subject the pharmaceutical composition according to claim 13.

15. A pharmaceutical kit comprising, in a single package, the combination according to claim 1, wherein the kit includes:
i) a first composition comprising the macrolide;
ii) a second composition comprising the immune checkpoint inhibitor; and
iii) instructions for use of the first and second compositions,
wherein the kit is suitable for use in the treatment of cancer.

16. A method of treating a viral disease in a subject in need thereof by activating or stimulating the immune system of the subject, comprising administering to the subject the pharmaceutical composition according to claim 13.

17. The combination according to claim 8, wherein the first and second pharmaceutical compositions are designed for different administration routes.

18. The combination according to claim 1, wherein the macrolide is: 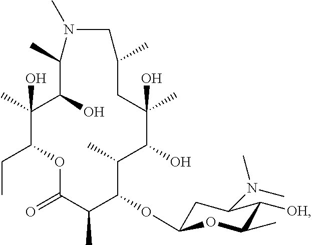
or a pharmaceutically acceptable salt thereof; and
the immune checkpoint inhibitor is selected from ipilimumab, pembrolizumab, nivolumab, atezolizumab, avelumab, and durvalumab.

19. A process for preparing a macrolide of Formula (I): 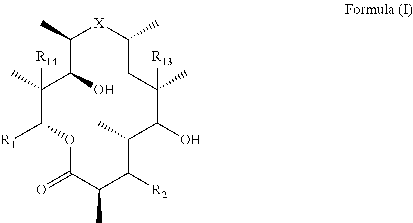
or a pharmaceutically acceptable salt thereof, wherein:
X is selected from —NR₃CH₂—, —CH₂NR₃—, —NR₃(C═O)—, —(C═O)NR₃—, and C═NOH;
R₂ is a sugar of Formula (II) or Formula (III): 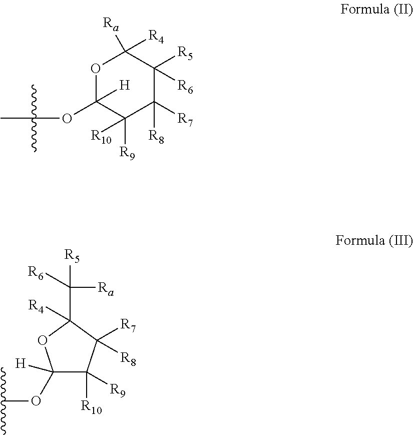
R₁ is selected from an alkyl, heteroalkyl, cycloalkyl, aryl, and heteroaryl moiety;
the alkyl moiety is selected from C₁-C₆ alkyl groups that are optionally branched;
the heteroalkyl moiety is selected from C₁-C₆ alkyl groups that are optionally branched or substituted and that comprise one or more heteroatoms;
the cycloalkyl moiety is selected from C₃-C₆ cyclic alkyl groups that are optionally substituted and that optionally comprise one or more heteroatoms;
the aryl moiety is selected from optionally substituted C₆ aromatic rings;
the heteroaryl moiety is selected from optionally substituted C₁-C₅ aromatic rings comprising one or more heteroatoms;
the heteroatoms are selected from O, N, P, and S;
the substituents, independently, are selected from alkyl, OH, F, Cl, NH₂, NH-alkyl, NH-acyl, S-alkyl, S-acyl, O-alkyl, and O-acyl; and
the acyl is selected from C₁-C₄ optionally branched acyl groups;
R₃ is selected from H and Me;
R₄ is selected from H and Me;
Rₐ is selected from H and CR₂₁R₂₂R₂₃;
R₂₁, R₂₂, R₂₃, and R₅, R₆, R₇, R₈, R₉, and R₁₀, independently, are selected from H, Me, NR₁₁R₁₂, NO₂, and OR₁₁;
R₂₃ together with R₄ in Formula (II), R₄ together with R₅ in Formula (II), R₅ together with R₇ in Formula (II), and R₇ together with R₉ in Formula (II), independently, may be joined to represent a bond to form a double bond between the carbon atoms that each group is connected to;
R₂₁ together with R₂₂, R₅ together with R₆, R₇ together with R₈, or R₉ together with R₁₀ may form a carbonyl;
R₁₁ and R₁₂, independently, are selected from H and alkyl;
R₁₃ is selected from H, OH, and OCH₃;
R₁₄ is selected from H and OH; and
one of R₅, R₆, R₇, R₈, R₉ or R₁₀ is selected from NR₁₁R₁₂ and NO₂;
the process comprising subjecting an aglycone of Formula (IV): 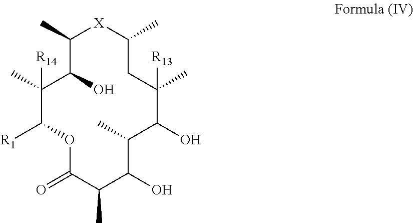
or a pharmaceutically acceptable salt thereof,
to a culture of a biotransformation strain which glycosylates the aglycone of Formula (IV) at the 3-hydroxyl position to form the macrolide of Formula (I).
